(19) United States
(12) Patent Application Publication
(10) Pub. No.: US 2006/0183991 A1
(43) Pub. Date: Aug. 17, 2006

(54) Polymorphisms in the th clcn7 gene as genetic markers for bone mass

(75) Inventor: Stuart Ralston, Aberdeenshire (GB)

Correspondence Address: BACON & THOMAS, PLLC, 625 SLATERS LANE, FOURTH FLOOR, ALEXANDRIA, VA, 22314, US

(73) Assignee: The University Court of the University of Aberdeen, Aberdeenshire (GB)

(21) Appl. No.: 10/535,914

(22) PCT Filed: Nov. 20, 2003
(86) PCT No.: PCT/GB03/05055

§ 371 Date: Mar. 6, 2006

(30) Foreign Application Priority Data
Nov. 21, 2002 (GB) 0227243.3

(51) Int. Cl. A61B 5/05

(52) U.S. Cl. 600/407

(57) ABSTRACT

Provided are genetic methods and materials for assessing bone mineral density (BMD) and determining the susceptibility of an individual to a disorder which is associated with a low level of BMD, the method comprising use of chloride channel 7 (Clcn7) marker. The methods may be used e.g. for diagnosis of osteoporosis. Preferred Clcn7 markers at specified positions are disclosed.

The present invention relates to methods for genetic analysis of bone mineral density and susceptibility to disorders which are related to bone mass. It further relates to materials for use in such methods.

### BACKGROUND ART

Genetic factors play an important role in the pathogenesis of osteoporosis—a common disease characterised by reduced bone mass, microarchitectural deterioration of bone tissue and increased susceptibility to fragility fractures (Kanis et al. 1994). Bone mineral density (BMD) is an important predictor of osteoporotic fracture risk and evidence from twin and family studies suggests that between 50%-85% of the variance in BMD is genetically determined (Gueguen et al. 1995; Arden and Spector 1997; Smith et al. 1973). However the genes responsible for these effects are incompletely defined. BMD is a complex trait, which is likely to be regulated by an interaction between environmental factors such as diet and exercise several different genes, each with modest effects on BMD.

A wide variety of candidate genes have been studied so far in relation to BMD, including the vitamin D receptor (Morrison et al. 1997), the estrogen receptor (Kobayashi et al. 1996), and the COLIAL gene (Grant et al. 1996). Current evidence suggests that allelic variation in these genes accounts for only a small portion of the variance in BMD however (Rubin et al. 1999) indicating that most of the genes which regulate BMD remain to be discovered.

The identification and genotyping of polymorphisms associated with regulation of BMD is useful, inter alia, in defining markers of bone mass and hence, for example, susceptibility to osteoporotic fractures.

### DISCLOSURE OF THE INVENTION

The present inventors have demonstrated that allelic variations in the CLCN7 gene contribute to regulation of bone mass in normal individuals.

The CLCN7 gene encodes an endosomal/lysosomal chloride channel (termed the ‘Chloride channel 7’) which is responsible for transport of chloride ions into the resorption lacuna. Here, they combine with hydrogen ions, to form hydrochloric acid which is responsible for dissolving hydroxyapatite crystals in mineralised bone (Vaananen et al. 2000). The CLCN7 gene maps to human chromosome 16p13 and comprises 25 exons. The CLCN7 gene product is highly expressed in the osteoclast ruffled border (Kornak et al. 2001). It is thought that the CLCN7 gene product forms functional dimers that pump chloride ions into the resorption lacuna.

Recent studies have shown that homozygous inactivating mutations of CLCN7 in mice and humans lead to severe osteopetrosis (Kornak et al. 2001). This is a condition characterised by increased bone mass because osteoclasts are unable to resorb bone normally (Janssens and Van Hul 2002). Other work has shown that heterozygous missense mutations of CLCN7 cause a milder form of the disease, termed autosomal dominant osteopetrosis type II, or Albers Schonberg disease (Cleiren et al. 2001). The missense mutations that cause ADO type 2 are thought to cause conformational changes in CLCN7 and exert dominant negative effects on chloride channel function. Known mutations in CLCN7 are listed in table 1.

However a role for the CLCN7 in regulating bone mass in normal individuals has not previously been taught.

Briefly, the present inventors conducted mutation screening of the CLCN7 gene in a cohort of 1032 individuals and identified several polymorphisms, several of which resulted in animo acid changes. These are summarised in Table 2. These were two missense polymorphisms in exon 1, and one missense polymorphism in exon 15, which caused amino acid changes. The inventors also demonstrated a significant association between BMD values and an allelic variant of the CLCN7 gene defined by a 50 bp tandem repeat polymorphism within intron 8 (Table 3). Specifically it was found that individuals carrying one or two alleles with 3 tandem repeats of this polymorphism had significantly higher spine BMD values that those who did not carry this variant. An association with femoral neck BMD was found with the G19240A and T19233C polymorphisms in exon 15 of the CLCN7 gene and BMD such that GG homozygotes at the G19240A site had higher BMD values that GA heterozygotes and AA homozygotes; and that TT homozygotes at the T19233C polymorphism had higher BMD values that TC heterozygotes and CC homozygotes.

Certain of the these mutations were discussed, after the priority date of the present application, in abstracts O-27 and P-354 of the 30^{th} European Symposium on Calcified Tissues (Rome, Italy, 8-12 May 2003).

Thus it appears that common allelic variants of the CLCN7 gene can account for at least part of the heritable component of BMD. Genotyping the CLCN7 intronic polymorphism or other polymorphisms may therefore be useful as genetic markers for BMD. This would be of clinical value e.g. in assessing the risk of osteoporosis and targeting preventative treatments.

### BRIEF DESCRIPTION OF THE INVENTION

At its most general, the present invention provides methods for assessing bone mass, and particularly BMD (e.g. lumbar spine BMD or femoral neck BMD) in an individual, the methods comprising using a CLCN7 marker, particularly a polymorphic marker to assess this trait.

In preferred embodiments these methods may be used to assess the susceptibility of the individual to disorders within the normal population which are to some extent (wholly or partly) related BMD—in particular disorders associated with low BMD, especially osteoporosis and related disorders. For example, the methods of the present invention may be used to determine the risk of certain consequences of relatively low BMD, such as to determine the risk of osteoporotic fracture (McGuigan et al (2001) Osteoporosis International, 12, 91-96). Such disorders are hereinafter termed “BMD-related disorders” and the methods and materials herein may also be used for the diagnosis and\or prognosis for them.

The method may comprise:

(i) providing a sample of nucleic acid, preferably genomic DNA, from an individual, and

(ii) establishing the presence or identity of one or more CLCN7 (polymorphic) markers in the nucleic acid sample, plus optionally one or more further steps to calculate a risk of osteoporosis or osteoporotic fracture in the individual based on the result of (ii).

Predicting Risk of Osteoporotic Fractures

The methods of the present invention may be used to attribute a likely BMD value to the individual based on the result established at (ii).

Alternatively or additionally they may be used in prognostic tests to establish, or assist in establishing, a risk of (developing an) osteoporotic fracture, which is the major clinical expression of osteoporosis. Methods for making such predictions are well known to those skilled in the art and the present disclosure may be used in conjunction with existing methods in order to improve their predictive power. Other known predictors include BMD, weight, age, sex, clinical history, menopausal status, HRT use, various SNPs and so on. The diagnosis of osteoporosis (and prognosis of fracture) is reviewed by Kanis et al (1994) J Bone and Mineral Res 9,8: 1137-1141.

McGuigan et al (2001) supra disclose predictive methods based on a combination of bone densitometry and genotyping (in that case COLIA1 genotyping). Individuals were classified as either high or low risk on the basis of these two methods, which were inter-related but independently predicted risk of sustaining osteoporotic fractures. Thus, by analogy, the present CLCN7 test may be predictive independently of BMD scores.

Marshall (1996) BMJ 312: 1254-1259 discloses a meta-analysis of how BMD measures predict osteoporotic fractures and attributed relative risk values and confidence intervals to various BMD measurements. The paper refers to a number of other risk factors for fracture. Cummings et al (1995) N Engl J Med 332: 767-73, also reviews risk factors (in that case for hip fracture in white woman).

All of these papers, inasmuch as they may be utilised by those skilled in the art in practising the present invention, are hereby incorporated by reference.

Thus preferred aspects of the invention will involve establishing or utilising one or more further measures which are predictive of osteoporotic fracture and defining a risk value (e.g. low, medium, high) or relative risk values or odds ratios (adjusted, for instance, against the population of that age and optionally sex) and optionally a confidence value or interval, based on the combination of these. Statistical methods for use in such predictions (e.g. Chi-square test, logistic regression analysis and so on) are well known to those skilled in the art. In a preferred embodiments a battery of tests (both genotyping and phenotyping) will be employed to maximise predictive power.

The methods may further include the step of providing advice to individuals characterised as being above low or medium risk, in order to reduce that risk (e.g. in terms of lifestyle, diet, and so on).

Particular methods of detecting polymorphisms in nucleic acid samples are described in more detail hereinafter.

Nucleic Acid Sample

The sample from the individual may be prepared from any convenient sample, for example from blood or skin tissue. The DNA sample analysed may be all or part of the sample being obtained. Methods of the present invention may therefore include obtaining a sample of nucleic acid obtained from an individual. Alternatively, the assessment of the CLCN7 polymorphic marker may be performed or based on an historical DNA sample, or information already obtained therefrom e.g. by assessing the CLCN7 polymorphic marker in DNA sequences which are stored on a databank.

Where the polymorphism is not intronic the assessment may be performed using mRNA (or cDNA), rather than genomic DNA.

Choice of Individual

Where the present invention relates to the analysis of nucleic acid of an individual, such an individual will generally be entirely symptomless and\or may be considered to be at risk from BMD-related disorder such as osteoporosis (e.g. by virtue of other determinants e.g. age, weight, menopausal status, HRT use etc. (see discussion above).

The method may be used to assess risk within a population by screening individual members of that population.

Preferred Markers

It is preferred that the polymorphic marker is a microsatellite repeat polymorphisms or a single nucleotide polymorphism (SNP), which may be in an intron, exon or promoter sequence of the CLCN7 gene. Preferably it will be a common polymorphism (allele frequency>0.05).

Preferred polymorphisms are as follows:

c39699g situated in exon 1.

g39705c situated in exon 1.

t39716c situated in exon 1.

14476 50 bp repeat polymorphism, situated within intron 8.

t19233c, situated in exon 15

g19240a, situated in exon 15.

It should be noted that c39699g, g39705c and t39716c are numbered in relation to the reverse complement of the sequence with accession number AL031705. The surrounding sequence is attached at Appendix I for reference. These polymorphisms were previously designated 40570 and 40576 and 40587 in accordance with earlier sequence accessions.

The 50 bp repeat polymorphism, and g19240a and t19233c are numbered in relation to the reverse complement of the sequence with accession number AL031600. The surrounding sequence is attached at Appendix II for reference.

Most preferred are polymorphisms are the SNPs at positions: c39699g, g39705c and the 50 bp repeat within Intron 8, commencing at nucleotides 14476. A significant association is found between lumbar spine BMD and number of tandem repeats within Intron 8. Specifically individuals carrying one or more alleles with 3 tandem repeats have increased BMD.

Also there is a significant association between the polymorphisms at positions 19240 and 19233 and femoral neck BMD Other SNP positions which may be used are listed in table 2.

Accordingly, in one embodiment the method of the present invention comprises assessing in a genomic DNA sample obtained from an individual one or more CLCN7 polymorphisms selected from the SNP's at the following positions:

39699, 39705, 39716, 19240 19233 and the 50 bp repeat within Intron 8, or a polymorphism in linkage disequilibrium with one of said polymorphisms.

In a further embodiment, the method may comprise assessing two, three, four or five of the CLCN7 polymorphisms. Any suitable combination of one or more markers may be used to assess the BMD trait. For example, the method may comprise assessing 19233, 19240 and the 50 bp repeat within Intron 8.

The method of the invention may comprise, in addition to assessing one or more CLCN7 polymorphisms, or one or more polymorphisms in linkage disequilibrium with a CLCN7 polymorphisms, the assessment of other polymorphisms which are linked or associated with a BMD-related disorder.

Examples of such other polymorphisms include polymorphisms in the VDR gene and the COLIA1 gene (Uitterlinden, et al. (2001) Journal of Bone and Mineral Research).

Identity of Alleles

The assessment of an SNP or microsattelite polymorphism will generally involve determining the identity of a nucleotide or nucleotides at the position of said polymorphism.

Preferred assessment of the SNPs at the positions described above will establish whether or not the individual is heterozygous or homozygous for the allele at these sites.

Preferred assessment of the microsattelite polymorphism within Intron 8 will establish whether or not the individual is heterozygous or homozygous for a specific length variant at this site (and hence high lumbar spine BMD). Individuals will 1 or 2 copies of the allele containing 3 repeats of the Intron 8 microsattelite were found to have higher spine BMD values that those without this length variant (see Table 6).

For example, for the 50 bp repeat polymorphism, in relation to likely susceptibility to a disorder associated with low spine BMD, an individual who is homozygous for alleles containing 3 repeats of the polymorphism is classified as being at the lowest risk; an individual who is heterozygous for alleles containing 3 repeats is classified as having intermediate risk; and an individual who has no alleles containing 3 repeats is in the higest risk category.

Microsatellite repeats are highly polymorphic and it is likely that the alleles containing 3 repeats are in linkage disequlibrium with other polymorphisms in the CLCN7 gene such as those at positions 39699, and 39705 in exon 1, or 19233 or 19240 in exon 15.

The lower statistical significance for the femoral neck BMD is not entirely surprising, since there is now good evidence from both human and animal studies to suggest that the effects of genetic factors on BMD regulation are specific to BMD sites (Koller et al. 2000; Stewart and Ralston 2000).

Use of Functional Polymorphisms

Most preferred for use in the present invention are SNPs which are directly responsible for the BMD phenotype (“functional polymorphisms”). Intronic SNPs may, for example, be situated in regions involved in gene transcripton. SNPs may be directly responsible for the BMD phenotype because of an effect on the amino acid coding, or by disruption of regulatory elements, e.g., which may regulate gene expression, or by disruption of sequences (which may be exonic or intronic) involved in regulation of splicing, such as exonic or splicing enhancers as discussed below.

Irrespective of these points and the precise underlying cause of the associations described herein, those skilled in the art will appreciate that the disclosure has great utility for genotyping of BMD in individuals, whether through functional polymorphisms, or polymorphisms which are in linkage disequilibrium with functional polymorphisms (which may be elsewhere in the CLCN7 locus or in other genes nearby). The invention thus extends to the use not only of the markers described above, but also (for example) to polymorphic markers which are in linkage disequilibrium with any of the markers discussed above.

Use of Other Polymorphisms

As is understood by the person skilled in the art, linkage disequilibrium is the non-random association of alleles. Further details may be found in Kruglyak (1999) Nature Genetics, Vol 22, page 139 and Boehnke (2001) Nature Genetics 25: 246-247). For example, results of recent studies indicate (summarised by Boehnke) that significant linkage disequilibrium extends for an average distance of 300 kb in the human genome.

Other polymorphic markers which are in linkage disequilibrium with any of the polymorphic markers described above may be identified in the light of the disclosure herein without undue burden by further analysis e.g., within the CLCN7 gene.

Thus in a related aspect, the present invention provides a method for mapping further polymorphisms which are associated, or are in linkage disequilibrium with a CLCN7 polymorphism, as described herein. Such a method may preferably be used to identify further polymorphisms associated with variation in BMD. Such a method may involve sequencing of the CLCN7 gene, or may involve sequencing regions upstream and downstream of the CLCN7 gene for associated polymorphisms.

In a further aspect, the present invention provides a method of identifying open reading frames which influence BMD. Such a method may comprise screening a genomic sample with an oligonucleotide sequence derived from a CLCN7 polymorphic marker as described herein and identifying open reading frames proximal to that genetic sequence.

A region which is described as ‘proximal’ to a polymorphic marker may be within about 1000 kb of the marker, preferably within about 500 kb away, and more preferably within about 100 kb, more preferably within 50 kb, more preferably within 10 kb of the marker.

Materials

The invention further provides oligonucleotides for use in probing or amplification reactions, which may be fragments of the sequences contained with accession numbers AL031705 and AL031600 or a polymorphic variant thereof (see Table 2 and appendices 1 & 2 herein).

Preferred primers are as follows:

For exon 1 SNP's
Forward TTGCAGGTCACATGGTCGGCCGTCGCTC
Reverse GACACGCGGCGCCGCAGAAGGCTCAC
For Intron 8 microsattelite
Forward CCACTCCAGCTGGAGCCTGAGG
Reverse GCTGAGGGAAGCCCATCTCC
For Exon 15 SNP:
Forward TTGCAGTGAGCCAAGATCGC
Reverse CTCCTCCCGTAGCCTAAGCG

These and other primer pairs used in mutation analysis and genotyping of CLCN7 are shown in Table 3.

Nucleic acid for use in the methods of the present invention, such as an oligonucleotide probe and/or pair of amplification primers, may be provided in isolated form and may be part of a kit, e.g. in a suitable container such as a vial in which the contents are protected from the external environment. The kit may include instructions for use of the nucleic acid, e.g. in PCR and/or a method for determining the presence of nucleic acid of interest in a test sample. A kit wherein the nucleic acid is intended for use in PCR may include one or more other reagents required for the reaction, such as polymerase, nucleosides, buffer solution etc. The nucleic acid may be labelled. A kit for use in determining the presence or absence of nucleic acid of interest may include one or more articles and/or reagents for performance of the method, such as means for providing the test sample itself, e.g. a swab for removing cells from the buccal cavity or a syringe for removing a blood sample (such components generally being sterile).

The various embodiments of the invention described above may also apply to the following: a diagnostic means for determing the risk of a BMD-related disorder (e.g. osteoporosis); a diagnostic kit comprising such a diagnostic means; a method of osteoporosis therapy, which may include the step of screening an individual for a genetic predisposition to osteoporosis, wherein the predisposition is correlated with a CLCN7 polymorphic marker, and if a predisposition is identified, treating that individual to prevent or reduce the onset of osteoporosis (such a method may comprise the treatment of the individual by hormone replacement therapy); and the use, in the manufacture of means for assessing whether an individual has a predisposition to osteoporosis, of sequences (e.g., PCR primers) to amplify a region of the CLCN7 gene.

Assessment of Polymorphisms

Methods for assessment of polymorphisms are reviewed by Schafer and Hawkins, (Nature Biotechnology (1998)16, 33-39, and references referred to therein) and include: allele specific oligonucleotide probing, amplification using PCR, denaturing gradient gel electrophoresis, RNase cleavage, chemical cleavage of mismatch, T4 endonuclease VII cleavage, multiphoton detection, cleavase fragment length polymorphism, E. coli mismatch repair enzymes, denaturing high performance liquid chromatography, (MALDI-TOF) mass spectrometry, analysing the melting characteristics for double stranded DNA fragments as described by Akey et al (2001) Biotechniques 30; 358-367. These references, inasmuch as they be used in the performance of the present invention by those skilled in the art, are specifically incorporated herein by reference.

The assessment of the polymorphism may be carried out on a DNA microchip, if appropriate. One example of such a microchip system may involve the synthesis of microarrays of oligonucleotides on a glass support. Fluorescently—labelled PCR products may then be hybridised to the oligonucleotide array and sequence specific hybridisation may be detected by scanning confocal microscopy and analysed automatically (see Marshall & Hodgson (1998) Nature Biotechnology 16: 27-31, for a review).

Some preferred examples of such methods will now be discussed in more detail.

Use of Nucleic Acid Probes

The method of assessment of the polymorphism may comprise determining the binding of an oligonucleotide probe to the nucleic acid sample. The probe may comprise a nucleic acid sequence which binds specifically to a particular allele of a polymorphism and does not bind specifically to other alleles of the polymorphism. Where the nucleic acid is double-stranded DNA, hybridisation will generally be preceded by denaturation to produce single-stranded DNA. A screening procedure, chosen from the many available to those skilled in the art, is used to identify successful hybridisation events and isolated hybridised nucleic acid.

Probing may employ the standard Southern blotting technique. For instance DNA may be extracted from cells and digested with different restriction enzymes. Restriction fragments may then be separated by electrophoresis on an agarose gel, before denaturation and transfer to a nitrocellulose filter. Labelled probe may be hybridised to the DNA fragments on the filter and binding determined.

Binding of a probe to target nucleic acid (e.g. DNA) may be measured using any of a variety of techniques at the disposal of those skilled in the art. For instance, probes may be radioactively, fluorescently or enzymatically labelled.

Polymorphisms may be detected by contacting the sample with one or more labelled nucleic acid reagents including recombinant DNA molecules, cloned genes or degenerate variants thereof under conditions favorable for the specific annealing of these reagents to their complementary sequences within the relevant gene.

As is understood by those skilled in the art, a ‘complement’ or ‘complementary’ or ‘reverse complement’ sequence (the terms are equivalent) is one which is the same length as a reference sequence, but is 100% complementary thereto whereby by each nucleotide is base paired to its counterpart running in anti-parallel fashion i.e. G to C, and A to T or U.

Preferably, the lengths of these nucleic acid reagents are at least 15 to 30 nucleotides. After incubation, all non-annealed nucleic acids are removed from the nucleic acid:gene hybrid. The presence of nucleic acids that have hybridized, if any such molecules exist, is then detected. Using such a detection scheme, the nucleic acid from the cell type or tissue of interest can be immobilized, for example, to a solid support such as a membrane, or a plastic surface such as that on a microtitre plate or polystyrene beads. In this case, after incubation, non-annealed, labeled nucleic acid reagents are easily removed. Detection of the remaining, annealed, labeled nucleic acid reagents is accomplished using standard techniques well-known to those in the art. The gene sequences to which the nucleic acid reagents have annealed can be compared to the annealing pattern expected from a normal gene sequence in order to determine whether a gene mutation is present.

Approaches which rely on hybridisation between a probe and test nucleic acid and subsequent detection of a mismatch may be employed. Under appropriate conditions (temperature, pH etc.), an oligonucleotide probe will hybridise with a sequence which is not entirely complementary. The degree of base-pairing between the two molecules will be sufficient for them to anneal despite a mis-match. Various approaches are well known in the art for detecting the presence of a mis-match between two annealing nucleic acid molecules. For instance, RN'ase A cleaves at the site of a mis-match. Cleavage can be detected by electrophoresing test nucleic acid to which the relevant probe or probe has annealed and looking for smaller molecules (i.e. molecules with higher electrophoretic mobility) than the full length probe/test hybrid. Other approaches rely on the use of enzymes such as resolvases or endonucleases.

Thus, an oligonucleotide probe that has the sequence of a region of the normal gene (either sense or anti-sense strand) in which polymorphisms associated with the trait of interest are known to occur may be annealed to test nucleic acid and the presence or absence of a mis-match determined. Detection of the presence of a mis-match may indicate the presence in the test nucleic acid of a mutation associated with the trait. On the other hand, an oligonucleotide probe that has the sequence of a region of the gene including a mutation associated with disease resistance may be annealed to test nucleic acid and the presence or absence of a mis-match determined. The presence of a mis-match may indicate that the nucleic acid in the test sample has the normal sequence, or a different mutant or allele sequence. In either case, a battery of probes to different regions of the gene may be employed.

As discussed above, suitable probes may comprise all or part of the sequence contained with accession numbers AL031705 and AL031600 (or reverse complement thereof), or all or part of a polymorphic form of these sequences (or reverse complement thereof (e.g. containing one or more of the polymorphisms shown in the Tables).

Those skilled in the art are well able to employ suitable conditions of the desired stringency for selective hybridisation, taking into account factors such as oligonucleotide length and base composition, temperature and so on.

Suitable selective hybridisation conditions for oligonucleotides of 17 to 30 bases include hybridization overnight at 42° C. in 6×SSC and washing in 6×SSC at a series of increasing temperatures from 42° C. to 65° C. One common formula for calculating the stringency conditions required to achieve hybridization between nucleic acid molecules of a specified sequence homology is (Sambrook et al., 1989): Tₘ=81.5° C.+16.6Log (Na+]+0.41 (% G+C)−0.63 (% formamide)−600/#bp in duplex.

Other suitable conditions and protocols are described in Molecular Cloning: a Laboratory Manual: 2nd edition, Sambrook et al., 1989, Cold Spring Harbor Laboratory Press and Current Protocols in Molecular Biology, Ausubel et al. eds., John Wiley & Sons, 1992.

Amplification-Based Methods

The hybridisation of such a probe may be part of a PCR or other amplification procedure. Accordingly, in one embodiment the method of assessing the polymorphism includes the step of amplifying a portion of the CLCN7 locus, which portion comprises at least one polymorphism.

The assessment of the polymorphism in the amplification product may then be carried out by any suitable method, e.g., as described herein. An example of such a method is a combination of PCR and low stringency hybridisation with a suitable probe. Unless stated otherwise, the methods of assessing the polymorphism described herein may be performed on a genomic DNA sample, or on an amplification product thereof.

Where the method involves PCR, or other amplification procedure, any suitable PCR primers may be used. The person skilled in the art is able to design such primers, examples of which are shown in Table 4.

An oligonucleotide for use in nucleic acid amplification may be about 30 or fewer nucleotides in length (e.g. 18, 21 or 24). Generally specific primers are upwards of 14 nucleotides in length, but need not be than 18-20. Those skilled in the art are well versed in the design of primers for use processes such as PCR. Various techniques for synthesizing oligonucleotide primers are well known in the art, including phosphotriester and phosphodiester synthesis methods.

Suitable polymerase chain reaction (PCR) methods are reviewed, for instance, in “PCR protocols; A Guide to Methods and Applications”, Eds. Innis et al, 1990, Academic Press, New York, Mullis et al, Cold Spring Harbor Symp. Quant. Biol., 51:263, (1987), Ehrlich (ed), PCR technology, Stockton Press, NY, 1989, and Ehrlich et al, Science, 252:1643-1650, (1991)). PCR comprises steps of denaturation of template nucleic acid (if double-stranded), annealing of primer to target, and polymerisation.

An amplification method may be a method other than PCR. Such methods include strand displacement activation, the QB replicase system, the repair chain reaction, the ligase chain reaction, rolling circle amplification and ligation activated transcription. For convenience, and because it is generally preferred, the term PCR is used herein in contexts where other nucleic acid amplification techniques may be applied by those skilled in the art. Unless the context requires otherwise, reference to PCR should be taken to cover use of any suitable nucleic amplification reaction available in the art.

Sequencing

The polymorphism may be assessed or confirmed by nucleotide sequencing of a nucleic acid sample to determine the identity of a polymorphic allele. The identity may be determined by comparison of the nucleotide sequence obtained with a sequence shown in the Annex, Figures and Tables herein. In this way, the allele of the polymorphism in the test sample may be compared with the alleles which are shown to be associated with susceptibility for osteoporosis.

Nucleotide sequence analysis may be performed on a genomic DNA sample, or amplified part thereof, or RNA sample as appropriate, using methods which are standard in the art.

Where an amplified part of the genomic DNA sample is used, the genomic DNA sample may be subjected to a PCR amplification reaction using a pair of suitable primers. In this way the region containing a particular polymorphism or polymorphisms may be selectively amplified (PCR methods and primers are discussed in more detail above). The nucleotide sequence of the amplification product may then be determined by standard techniques.

Other techniques which may be used are single base extension techniques and pyrosequencing.

Mobility Based Methods

The assessment of the polymorphism may be performed by single strand conformation polymorphism analysis (SSCP). In this technique, PCR products from the region to be tested are heat denatured and rapidly cooled to avoid the reassociation of complementary strands. The single strands then form sequence dependent conformations that influence gel mobility. The different mobilities can then be analysed by gel electrophoresis.

Assessment may be by heteroduplex analysis. In this analysis, the DNA sequence to be tested is amplified, denatured and renatured to itself or to known wild-type DNA. Heteroduplexes between different alleles contain DNA “bubbles” at mismatched basepairs that can affect mobility through a gel. Therefore, the mobility on a gel indicates the presence of sequence alterations.

Restriction Site Based Methods

Where an SNP creates or abolishes a restriction site, the assessment may be made using RFLP analysis. In this analysis, the DNA is mixed with the relevant restriction enzyme (i.e., the enzyme whose restriction site is created or abolished). The resultant DNA is resolved by gel electrophoresis to distinguish between DNA samples having the restriction site, which will be cut at that site, and DNA without that restriction site, which will not be cut.

Where the SNP does not create or abolish a restriction site the SNP may be assessed in the following way. A mutant PCR primer may be designed which introduces a mutation into the amplification product, such that a restriction site is created when one of the polymorphic variants is present but not when another polymorphic variant is present. After PCR amplification using this primer (and another suitable primer), the amplification product is admixed with the relevant restriction enzyme and the resultant DNA analysed by gel electrophoresis to test for digestion.

The invention will now be further described with reference to the following non-limiting Example, Tables and Annex. Other embodiments of the invention will occur to those skilled in the art in the light of these.

Examples of BMD-Related CLCN7 Polymorphisms

Subjects

The study group comprised 1032 women aged 45-55 who were randomly selected from a large population based BMD screening programme for osteoporotic fracture risk (Garton et al. 1992; Garton et al. 1992). The screening program involved 7000 women who were identified using Community Health Index records (CHI) from a 25-mile radius of Aberdeen, a city with a population of 250,000 in the North East of Scotland. Women were invited by letter to undergo BMD measurements between 1990-1994 and 5000 of the 7000 invited (71.4%) attended for evaluation. Blood samples were subsequently obtained for DNA extraction on 81% (n=4050) of these individuals.

Participants were weighed wearing light clothing and no shoes on a set of balance scales calibrated to 0.05 kg (Seca, Hamburg, Germany). Height was measured using a stadiometer (Holtain Ltd, Crymych, United Kingdom). All participants gave written informed consent to being included in the study, which was approved by the Grampian University Hospitals Joint Ethical Committee.

Bone Mineral Densitometry

The bone mineral density measurements (BMD) of the left proximal femur (the femoral neck, FN) and lumbar spine, LS (L2-4) were performed by dual energy x-ray absorptiometry using one of two Norland XR26 or XR36 densitometers (Norland Corp, Wisconsin, USA). Calibration of the machines was performed daily, and quality assurance checked by measuring the manufacturer's lumbar spine phantom at daily intervals and a Hologic spine phantom at weekly intervals. The in-vivo precision for the XR36 was 1.2% for the lumbar spine (LS), and 2.3% for the femoral neck (FN). Corresponding values for the XR26 were 1.95% and 2.31% (LS and FN respectively).

Mutation Screening and Genotyping

Mutation screening was carried out by DNA sequencing of the promoter and intron exon boundaries of the CLCN7 gene (accession numbers AL031705 and AL031600) in DNA extracted from peripheral venous blood samples from about 50 individuals. This resulted in the identification of several polymorphisms as shown in table 1. Genotyping for the Intron 8 microsattelite polymorphisms was carried out using the following primer pairs:

Forward CCACTCCAGCTGGAGCCTGAGG
Reverse GCTGAGGGAAGCCCATCTCC

Genotypes were determined by agarose gel electophoresis followed by ethidium bromide staining.

Statistical Methods

Statistical analysis was carried out using Minitab version 12. On exploratory analysis, individuals carrying 3 repeats of the polymorphism within Intron 8 were found to have higher BMD values than individuals with other length variants. In view of this we coded patients by the presence or absence of allele 3 of the Intron 8 polymorphism. Differences in unadjusted BMD values between carriers of allele 3 genotypes were initially tested by ANOVA. We also used a General Linear Model analysis of variance (ANOVA) adjusting for height, weight, and age to study the contribution of the intron 8 VNTR allele 3 to regulation of BMD. The same procedure was used to test for allelic associations in relation to the T39716C polymorphism in exon 1 and the G19240A and T19233C polymorphisms within exon 15.

Results

Details of age, height, weight and BMD values in the whole study population are shown in Table 5.

The relationship between intron 8 microsatellite genotype and BMD values are shown in tables 6. There was a trend for a difference in spine BMD between genotype groups when subjects were categorised according to the presence or absence of 3 repeats of the Intron 8 50 bp repeat. The result was not significant using unadjusted BMD values, but was statistically significant when the values were adjusted for relevant covariates that influence BMD (Table 6). There was also a significant association between femoral neck BMD, adjusted for weight, height, menopausal status and age and the polymorphisms in exon 15 (g19240a and t19233c). The results of this are shown in table 7, which shows that individuals carrying two copies of the G allele at position 19240 have significantly higher BMD values than the other genotype groups. Also, individuals carrying two copies of the T allele at position 19233 have significantly higher BMD values than the other genotype groups. We found no association between the t39716c polymorphism and BMD.

TABLE 1
CLCN7 mutations associated with osteopetrosis

Codon affected
G215R
P249L
R286W
Q555X
R762Q
R765B
L766P
R767W
DelL688
2423delAG (frameshift)

Data from Cleiren (Cleiren et al. 2001) and Kornak (Kornak et al. 2001).

TABLE 2
Polymorphisms of the CLCN7 gene identified by mutation
screening of coding exons and intron-exon boundaries

| | | Amino acid (aa) | Sequence ID |
|---|---|---|---|
| Region | polymorphism | change | (accession no) |
| Exon 1 | c39699g | Leu37Val | AL031705 |
| | g39705c | Gly39Arg | |
| | t39716c | Pro42Pro | |
| Intron 1 | c6582t | | AL031600 |
| | c6594t | | |
| | c6682a | | |
| Exon 3 | g10428t | None | |
| Intron 3 | c10545a | | |
| Intron 4 | g10725a | | |
| Exon 5 | g11187c | None | |
| Intron 5 | c11463t | | |
| | a11530c | | |
| | t11559c | | |
| Exon 7 | c12974t | None | |
| | c12999t | None | |
| Intron 7 | a14319g | | |
| Intron 8 | 50bp repeat | | |
| | 14476-14726 | | |
| Intron 9 | t14859c | | |
| Exon 10 | g15967a | None | |
| Exon 13 | g17660a | None | |
| Intron 13 | t18080c | | |
| Exon 14 | a18218t | None | |
| Intron 14 | g19150a | | |
| | g19153a | | |
| Exon 15 | t19233c | None | |
| | g19240a | Val418Met | |
| Intron 16 | insertion | | |
| | g21387 | | |
| Exon 17 | g21596a | None | |
| Intron 19 | a23148g | | |
| | g23322a | | |
| Intron 20 | a23525g | | |
| | t23577c | | |
| | a23587g | | |
| | t23588g | | |
| | c23596t | | |
| Intron 21 | c24344t | | |
| Intron 22 | c24457t | | |
| Intron 23 | g24960a | | |

TABLE 3

Tandem 50 bp repeat polymorphism in intron 8
of CLCN7 gene

50 bp Repeat unit
(gtgtctctgagcaccggtccttctggtctccaggaagggccgcgtcacg
c) n

(n can vary from 3 to 9)

The table shows the sequence of the 50 bp repeat within intron 8 of the CLCN7 gene.

TABLE 4

Primers used for CLCN mutation screening and
genotyping Clcn7 primers

| | |
|---|---|
| EX1F° | TTGCAGGTCACATGGTCGGCCGTCGCTC |
| EX1R° | GACACGCGGCGCCGCAGAAGGCTCAC |
| EX2F | TCTAGAGCAGGGAGCTTGCG |
| EX2R | GCCCTGGGGCCCCACTATCT |
| EX3-4F | CCTTGGTGTCGGGATGATAA |
| EX3-4R | GGAGTCAGAGGAGGAGGGAG |
| EX5-6F | GCACACTGGGCCCTTCATAA |
| EX5-6R | TTCACCAAGACCCCCAATCC |
| EX7F | GCTGAGGGGCTGCATCTGTC |
| EX7R | AAGGCAGGCAGCCAAGAGAG |
| EX8-9F | CAGCCACTCTGCCTGATCGG |
| EX8-9R | AGGCTGTCCTCAGATGGGGC |
| EX10-11F | TCAGAGCTGCTGACTCGGTT |
| EX10-11R | AGGACCAAGGCCTGACAGAC |
| EX12F | TCCCCTCTTGCTCTCCACTG |
| EX12R | CTCAACCTGGGCCTTAAGCA |
| EX13-14F | AAGGAGCTGTGGGCCTTTTC |
| EX13-14R | GTGGCCTAGGAGTGTAAACC |
| EX15F | TTGCAGTGAGCCAAGATCGC |
| EX15R | CTCCTCCCGTAGCCTAAGCG |
| EX16F | CTCATCTCCCCTCCCAACGT |
| EX16R | CCTCCTGCCTTGGTCTCTCC |
| EX17F | CTGGAAGGTGACTGTGAGGC |
| EX17R | TGAACCACGTGAGGTGCGAC |
| EX18-19F | TCTGTGTATCTTGGTGGGTT |
| EX18-19R | GGGAACAGAGGGCTTGAGGA |
| EX20-21F | GGGGTAGGCTCAGGGTTTCT |
| EX20-21R | CCCACCAATGGACTCGACAG |
| EX22-23F | CATGCCCAGATGGGAAATCT |
| EX22-23R | CCCGGAACAGCTTGAACACC |
| EX24-25F | GGGCCTGGCAGGCTTTAGAG |
| EX24-25R | TCCGGGAGGAAATGCAGAAG |

°5% DMSO

TABLE 5
Demographics of study population

| | |
|---|---|
| Number | 1023 |
| Age | 47.6 ± 1.42 |
| Spine BMD (g/cm²) | 1.049 ± 0.14 |
| Femoral Neck BMD (g/cm²) | 0.876 ± 0.11 |
| Weight | 64.9 ± 11.4 |
| Height | 160.6 ± 11.6 |

TABLE 6
Association between CLCN7 microsatellite
genotypes and BMD values

| Copies of allele 3 | | N | LS BMD | FN BMD |
|---|---|---|---|---|
| 0 | (unadjusted) | 443 | 1.047 ± 0.153 | 0.885 ± 0.115 |
| | (adjusted) | | 1.048 ± 0.007 | 0.886 ± 0.005 |
| 1 | (unadjusted) | 448 | 1.063 ± 0.149 | 0.889 ± 0.123 |
| | (adjusted) | | 1.062 ± 0.007 | 0.888 ± 0.005 |
| 2 | (unadjusted) | 129 | 1.082 ± 0.151 | 0.887 ± 0.121 |
| | (adjusted) | | 1.083 ± 0.013 | 0.889 ± 0.010 |

| | | |
|---|---|---|
| p-value | | |
| (unadjusted) | 0.067 | 0.889 |
| (adjusted) | 0.036 | 0.933 |
| (ANOVA) | | |

BMD Values shown are mean±standard deviation, either unadjusted, or adjusted for age, weight, height, menopausal status and HRT use, by GLM ANOVA. P-values shown are for differences between genotype

TABLE 7
Association of adjusted BMD with
exon 15 CLCN7 polymorphisms

| | N/(%) | Spine BMD | Hip BMD |
|---|---|---|---|
| T19233T | 712 (78.5%) | 1.064 ± 0.005 | 0.896 ± 0.004 *** |
| T19233C | 180 (19.8%) | 1.044 ± 0.010 | 0.863 ± 0.008 |
| C19233C | 12 (1.7%) | 1.032 ± 0.036 | 0.868 ± 0.028 |
| G19240G | 709 (78.2%) | 1.063 ± 0.005 | 0.895 ± 0.004 *** |
| G19240A | 184 (20.3%) | 1.043 ± 0.010 | 0.867 ± 0.008 |
| A19240A | 14 (1.5%) | 1.058 ± 0.037 | 0.874 ± 0.029 |

BMD values are means ± SD, adjusted for weight, height, age and menopausal status
*** p < 0.0001 compared with the other genotype groups

### REFERENCES

1. Arden NK and Spector TD (1997) Genetic influences on muscle strength, lean body mass, and bone mineral density: a twin study. J Bone Miner Res 12 (12):2076-2081.
2. Cleiren E, Benichou O, Van Hul E, Gram J, Bollerslev J, Singer FR, Beaverson K, Aledo A, Whyte M P, Yoneyama T, devernejoul M C, and Van Hul W (2001) Albers-Schonberg disease (autosomal dominant osteopetrosis, type II) results from mutations in the ClCN7 chloride channel gene. Hum. Mol. Genet. 10 (25):2861-2867.
3. Garton M J, Torgerson D J, Donaldson C, Russell I T, and Reid D M (1992) Recruitment methods for screening programmes: trial of a new method within a regional osteoporosis study. Br Med J 305 (6845):82-84.
4. Grant S F A, Reid D M, Blake G, Herd R, Fogelman I, and Ralston S H (1996) Reduced bone density and osteoporosis associated with a polymorphic Sp1 site in the collagen type 1 alpha 1 gene. Nature Genetics 14:203-205.
5. Gueguen R, Jouanny P, Guillemin F, Kuntz C, Pourel J, and Siest G (1995) Segregation analysis and variance components analysis of bone mineral density in healthy families. J Bone Miner Res 12:2017-2022.
6. Janssens K and Van Hul W (2002) Molecular genetics of too much bone. Hum. Mol. Genet. 11 (20):2385-2393.
7. Kanis J A, Melton L J, III, Christiansen C, Johnston C C, and Khaltaev N (1994) The diagnosis of osteoporosis. J Bone Miner Res 9 (8):1137-1141.
8. Kobayashi S, Inoue S, Hosoi T, Ouchi Y, Shiraki M, and Orimo H (1996) Association of bone mineral density with polymorphism of the estrogen receptor gene. J. Bone Miner. Res. 11 (3):306-311.
9. Koller D L, Econs M J, Morin P A, Christian J C, Hui S L, Parry P, Curran M E, Rodriguez L A, Conneally P M, Joslyn G, Peacock M, Johnston C C, and Foroud T (2000) Genome Screen for QTLs Contributing to Normal Variation in Bone Mineral Density and Osteoporosis. J Clin Endocrinol Metab 85 (9):3116-3120.
10. Kornak U, Kasper D, Bosl M R, Kaiser E, Schweizer M, Schulz A, Friedrich W, Delling G, and Jentsch TJ (2001) Loss of the ClC-7 chloride channel leads to osteopetrosis in mice and man. Cell 104 (2):205-215.
11. Morrison N A, Qi J C, Tokita A, Kelly P, Crofts L, Nguyen T V, Sambrook P N, and Eisman J A (1997) Prediction of bone density from vitamin D receptor alleles (Erratum). Nature 387:106.
12. Rubin L A, Hawker G A, Peltekova V D, Fielding L J, Ridout R, and Cole D E (1999) Determinants of peak bone mass: clinical and genetic analyses in a young female Canadian cohort. Journal of Bone & Mineral Research 14 (4):633-643.
13. Smith D M, Nance W E, Kang K W, Christian J C, and Johnston C C (1973) Genetic factors in determining bone mass. J Clin Invest 52:2800-2808.
14. Stewart T L and Ralston S H (2000) Role of genetic factors in the pathogenesis of osteoporosis. J. Endocrinol. 166 (2):235-245.

15. Vaananen H K, Zhao H, Mulari M, and Halleen J M (2000) The cell biology of osteoclast function. J. Cell Sci. 113:377-381.

APPENDIX 1
extract of reverse complement of sequence accession AL031705

!·NA_SEQUENCE 1.0
REVERSE-COMPLEMENT of: a1031705.em_hum check: 3153 from: 1 to: 42569
ID HS305C8 standard; genomic DNA; HUM; 42569 BP.
AC AL031705;
SV AL031705.25
a1031705.rev Length: 4.2569 Nov. 14, 2003 18:33 Type: N Check: 4047
. . .
39551 CAGCCGGCGC TTCCCGGCCG GTGTCGCTCC GCGGCGGGCC ATGGCCAACG
39601 TCTCTAAGAA GGTGTCCTGG TCCGGCCGGG ACCGGGACGA CGAGGAGGCG
39651 GCGCCGCTGC TGCGGAGGAC GGCGCGGCCC GGCGGGGGGA CGCCGCTGCT
39701 GAACGGGGCT GGGCCTGGGG CTGCGCGCCA GGTGAGGCCG GGCAGGGCGC
39751 AGGCGGGGAA ACTGAGCCCT CGTGCGCCCC GCAGCCCGCG CCCTCGTGAG
39801 CCTTCTGGCG GCGCCGCGTG TCTCGGTCCT GGAGGCGACC GAGGCGCGGT
39851 GGACTCGGGA ACGCGCCCCG GGGCTCCTCG GCGGGGCCGG GCTGGCGGGG

APPENDIX 2
extract reverse complement of sequence accession AL031600

!!NA_SEQUENCE 1.0
REVERSE-COMPLEMENT of: a1031600.em_hum check: 1339 from: 1 to: 31513
ID H5390E6 standard; genomic DNA; HUM; 31513 BP.
AC AL031600;
SV AL031600.4
a1031600.rev Length: 31513 Nov. 14, 2003 18:03 Type: N Check: 8418
. . .

| | |
|---|---|
| 6401 | AGGATGGCCC AGGGTGCTGT GGCGGGCACT GCATTGGGGG CGGCGTGTTG |
| 6451 | TCCAGCCCTT CTTTCCTGGT GGGTGGCAGG TGCCTCGCTT TCAGTCTAGA |
| 6501 | GCAGGGAGCT TGCGCCCTGG ACTCGGGCTG GACGTGTCGC TGACAGGCCG |
| 6551 | AGGGGCAGCC GGATCAGTTC TGCTTCCAGG GCCCAGGGAG GCCCGTCCCA |
| 6601 | GCCCTGCTGC CCCCACCCAG CAGGCAGGCC TGGCCTAGCC CATTCCTGAG |
| 6651 | CTCCCGGGCA GGGTCAGGCG AGGCCAGGGT GCGGCGGCGG GAGTGAGAAT |
| 6701 | CCACGGAGCA GAGCGTGCGA CGCCTGAGCG CCCTCATGAT TTCTCTTCTG |
| 6751 | CTTTTAGTCA CCACGTTCTG CGCTTTTCCG AGTCGGACAT ATGAGCAGCG |
| 6801 | TGGAGCTGGA TGATGAACTT TTGGACCCGG TGAGTTGGGG GTGTTCCCCG |
| 6851 | TCCTCCCGCA GAGCTAGCTG CATCTTAGCA GAGGGTGACA GGGATGGGCA |
| 6901 | CGGGCCGAGC GGCAGGGAGA TAGTGGGCCC CCAGGGCCGG GGTTCAGGGA |
| 6951 | AGATTTCCTT GGGGGGACAT GGTCCCTGAC GCCAACTGAG CAGAGGCAGC |
| 7001 | TGGGCAGAAG TGCTCTCAGA CGGAGGAGTG CAGGGCGCAG GAAGCCGGTC |
| 7051 | AGGACAGCAG TGACAGCATG GGCAGCGAGG GGGCTGGACC TGGCTTTGGG |
| 7101 | ACAGGGCAAG GACAGGGATC TTGGGGGGGC AGTGAGGAGC CCCAGGAGAG |
| 7151 | TGAGAGGGGG CCGGATGCCT CTGACTTCAG AGGGCAGGGG TTTAGATGTT |
| 7201 | CCCGTGCCAG TGGCTGCCCT GGGAGTCCTG AGCTCAGCGG CAGCGTGCTC |
| 7251 | GTCTTCCTTC CCCTCGGGGG CATCTCCCGC CGGCCTCGGT TTTTCCCCCA |
| 7301 | GCCGCTGGTG AGGCCGGGAG TCCTCTGCTG CCGCTGGCCG TTCACTCATC |
| 7351 | GTCTCTGGGT AGATGTCTGT GCGGGACTCC TGTTGAGATG ATCCTGATGT |
| 7401 | TGGCAACACC CCGGGCGTCC TCCTTCTCCC CATCAGGCCC CACCTGGCTC |
| 7451 | TGCCCTGGGC CACGTCAGAG GCTGAGGCAT CTCACAGTCC ACCTGTCCGG |
| 7501 | GTGCTCTTCG GCCTTGCGTC CGTTTGAGCT CTGCCGCAGT CGCTCCCGAG |
| 7551 | GCCGGCGCCG TGCTCAGATG CCGTCCTGTA CAGCCAGCAG CGCCTCTTCC |
| 7601 | GGGGCTGCCC TTCTGATACG TTTGTGCTGC CTCTGGAGCC ACAAGGCCTT |
| 7651 | CGGAAGATCT GTTTCGTGGC CGTGGGCGCC TTCGGCACTG CCTTTTTGGA |
| 7701 | CTTCAAAGCC TTTGCTCTGG TGTCAGCTTT GGGAGGGGCA GGAGTTGGGA |
| 7751 | GAGAAGGGAA AAAGCCAGCA CGTGAGATTC AGCAATCAGT CCTCTCCTGT |
| 7801 | CTCAACCCTG GAGCGGGTGC CTGGCCGGCC ACACGCGTGT TGGTTATGCT |
| 7851 | CATTTTTAAA CTGGGTTTGT TGTCTTTATA ATTGAGCTGC AGGAGTTCTT |
| 7901 | TATACATAGA TGCAAATCTC TCATCCAATA CATGATTTAT AGAAGTTTTC |
| 7951 | TCCCGTTCAG TGGGTTTTCT GTTCACTTTC TCAGTGGTGT CTTTTGTTGC |
| 8001 | TCAAATTTAT TTAATTAAAA AAGTTTTGGC CAAGGGAGGT GATTCGTGCC |
| 8051 | TGTAATCCTA GTACTTTGGG AAGCAGATGG ATTCATTGAG CTCAGGAGTT |
| 8101 | CAAGATCAGC CTGATCAACA TGGTGAAACC CTGTCTCTAC AAAAAATATA |
| 8151 | AATATTAGCT GGGCCTGGTG ATAGGCACCA GTAGTCCCAG CTACTTGGGA |
| 8201 | GGCTGAGGTT GGAGGATCAC TTGAGCCCAG GAGGTGGAGG TTTCAGTGAG |
| 8251 | CTGAGATGGT GCCACTGCAC TTCAGCCTGG GTGACAGAGT GAGATCCTGC |
| 8301 | CTCAAATTTT TTTTTTTTTT TCTGGGCAGG TGTGGTGGTT CACACCTGTA |
| 8351 | ATCCCAACAC TTTGGGAAAC CAAGGCTGCA GCCCAGGATT TGGAGATCAG |
| 8401 | CCTAGACAAC ACAGTGAGAC CCTGTCTCTA CAAAAAACAA AAACAAAAAC |
| 8451 | GAAAATTAGC CAGGTGTGGT GGTGTGCGCC TGTGGTCCCA GCTACTCAGG |
| 8501 | ACGCTGAGGC AGGTGGATTG ATCGAACCCA GGAGGTTGAG GCTGCAGTGA |
| 8551 | GCCATGATCA CACCATTGTA CTTCAGCCTG CGTGACAGAC GGGACCCTGT |
| 8601 | CTAAAAAAAT TAATTATTAC TATTCTTTGA GATGAGGTCT CACTGTGTGG |
| 8651 | CCCAGGCTGA ACTCCATCTC TCAGGCTCAA GCAATCCTCC CGTTTCAGCT |
| 8701 | TCTTCCTGAG GAGCTGGGAC CACAGGTGCA TCACACCCCG CACAGGTTGT |
| 8751 | ATTGCTGAGG TTCAGCTAAT CTGTTTTTTC TTGTGTTGCT TGTACTTTTG |
| 8801 | GTGTCAAATC TAAGAAACCA TTGCCTCACC CAAGAGTATG ACGACTGACC |
| 8851 | CGTTTTTTCC TAAGAATTTT ACAGTTTTAG GTCTTTCATC CCTTTTGAGT |
| 8901 | TAATTTTTGG ATGTGGTGTG AGGTAAGGGT CCAACGTCAT ACCCTCCCTC |
| 8951 | TCTCTCTCTC TTTTTTTGAG ACAGGGTCTC ACTGTCACCC AGGCTGGAGT |
| 9001 | GCAGTGGTGC AATCATGGTT CACTGCAGCC TCTGCCTCCT GTCTGTCTCC |
| 9051 | CAAGTAGCTG GGACTCAGGC GCATGTCACC ATACTCAGCT AATATTTTGT |
| 9101 | AGAGATGGAG TCTTACTATG TTGCCCAGGC TGATCACAAA CTCCTGGCCT |
| 9151 | CAAGCAGTCC TTCTGCCTCT GCCTCCCAGA GTGCTGGGAT TATAGCTGTC |
| 9201 | AGCCATTGCG CCCGGCCCAG CTTCATTTTT GCATGTGGAA ATCCAGTTGT |
| 9251 | ACCAGCACCA TTTGTTGAAA ACACTACCTT TCTCTGTTGA AATGTTTTGA |
| 9301 | CACTGTTGTG GGAAATCAAT TGATCGTACA TGTTTTGGAT TTCTTTCTGG |
| 9351 | ACTCTCTCAA TTCTCTTCCA TTCTTTTGTG GCCATCTTCA TGCCAGTACC |
| 9401 | ATGCCTGGTT TTTTTTTTTT TTTTTTTTTT GGCTTTTTTT TAAGAGTTGG |
| 9451 | GGTCTCACTG TGTTGCCCAG GCTGGGTGGA TCACTTGAGG CCAAGAGTTT |
| 9501 | GAGACCAGCC TGGCCAACAT GGTGAAACCC CGTCTCTACT AAAGATACAA |
| 9551 | AAATTAGCCA GGCGTGGTGG TGCACACCTG TAATCCCAGC TACTTGGGAG |
| 9601 | GCTGAGGCAG GAGAATGGCT TTAACCTGGA AGGCGGAGGT TGCAGTGAGT |
| 9651 | TGAGATCGCG TCACTGCACT CTAGCCTGGG CAAAAAGAGT GACTGTATCT |
| 9701 | CAAAAAAAAA AAAAAAAAAA AAAAAAAAAA GACAGATGAG GGTTTTACTC |
| 9751 | TGTTGCCCAG GCTGGTCTTG AACTCCTGGC TTCAGTTGAT CCTCTTGCCT |
| 9801 | CTGCCTCCCA GAGTGCTGGG ATTACAGGTG TGAGCCACCG CACCCGGCCT |
| 9851 | CATGGATTGA TTTTTGGATG TTAAACTAAC TTGTATTCCT AGGCTGAATT |
| 9901 | CACCTTGCTC CTGGCATTGC TGGAATCACT TTGCTTGTGT CTTACCAAAG |
| 9951 | ATCTTTGCAT CCGTGGTTGT AGGGGTGTTG GTCTGTAGTT CTCTTTTTTT |
| 10001 | TTTTTTTCTT TGAGACGGAG TCTTGCTCTG TCACCCAGGC TGGAGTGCAA |
| 10051 | CGGCGCAATC TCGGCTCACT GCAACCTCTG CATCCCGGGT TCAAGCGATT |
| 10101 | CTCCTGCCTC AGCCTCCTGA GTAGCTGGGA TTACAGGCGC COACCACCAC |
| 10151 | GCCCAGCTAA TTTTTGTATT TTTAGTAGCG ACAGGGTTTC ATCTTGTTGT |
| 10201 | CCAGGCTGGT CTCGAACTCC TGACCGCAGC TGGTCCACTT GCCTCGGCCT |
| 10251 | CCCAAAGTGC TGGGATTGTA GGTGTCAGCC ACCGCGCCCC ATGTGCAGTT |
| 10301 | CTCTTGCTGT GTCCTTGTCC TTGGTGTCGG GATGATAATG GCCTCGTGTG |
| 10351 | TGAGCTGAGA GGGGCCTCTC TCCTTGTGGC CTTGTCAACT GTGCTTCTCT |
| 10401 | CTTTGCCTTT TTCTGCCACA GGATATGGAC CCTCCACATC CCTTCCCCAA |
| 10451 | GGAGATCCCA CACAACGAGA AGCTCCTGTC CCTCAAGTAT GAGGTGGGCG |
| 10501 | TCCTTCTGTC CCCCTGACCC TGAGACCCGG CCTCTGCCCC CTGCCAGCCC |
| 10551 | ACTCCCGGTC CCCTGTGCCC GCACCCAGAG CGTGGGTTCG GTGCTGAGTG |
| 10601 | CTGCCCTTGC TGTCCCGGCC TGCAGAGCTT GGACTATGAC AACAGTGAGA |
| 10651 | ACCAGCTGTT CCTGGAGGAG GAGCGGCGGA TCAATCACAC GGTGAGCTGG |
| 10701 | ACGCCGCTCC CTGCAGGGCC CCACGGTGCG GGGCCTGGTG CCGGCCGGGC |
| 10751 | CTGGGGCTGC TCTTCTGCCG GGGTGAGGTG ACGCACCTCC TCCCTCCTCC |
| 10801 | TCTGACTCCG CCTCTGAGGC CTGTGGTTCG TCTGGTTTCT AGAGACAGTG |
| 10851 | GGAGGGTCAC GGTCACCGTA ACCAAGAAGG CTGCTCTTAC GGCCGCCAGA |
| 10901 | TGCGGTGCCC AGCATAACAA CCGCTGGCTG TGAAGTTGTT GGGAATTCAC |
| 10951 | CCACCTCCCC GAGTCACCCT CGGGCCCCGG GTGCGCCTCA GATGTTGGCC |
| 11001 | AGAAACTGTC CTTTGTGGGA CTCAGCGCAC CGTGCACACT GGGCCCTTCA |
| 11051 | TAATCCCGGG GCCTGCAGGC GGTCTGGGCG GTCCTGCTGC TGCCAGAGTG |
| 11101 | ACTGCGCCAG GGCCCTGCCT GACCCTCGCC CTGACCGCGC CCTGCAGGCC |
| 11151 | TTCCGGACGG TGGAGATCAA GCGCTGGGTC ATCTGCGCCC TCATTGGGAT |
| 11201 | CCTCACGGGC CTCGTGGCCT GCTTCATTGA CATCGTGGTG GAAAACCTGG |
| 11251 | CTGGCCTCAA GTACAGGGTC ATCAAGGGCA GTATCCTTCC CAGTGCGGCC |
| 11301 | GCTGCAGCTT GGGAGGGGGG CGTGGCCTGG GCCGAGTCCC GGGCAGAAGT |
| 11351 | CCTGAGCCCA GCGTGTTCCA GTGCAGGTGG AGGCGGCCCG GCCAGGCTGG |
| 11401 | CTGTGTCCCT GTCATGGTTG GGCCGTGAGA CGTCTCTGGG ATGTCCAGTG |
| 11451 | AACATCATGG CTCCACCCAG CAGGGTGGCA TCTGCCAGGC TGGTCTGTGG |
| 11501 | GGCAGGGCTG AGGTCTGGGC TGGGTGGTCA TGACGGGGAA GCAGCCAGCC |
| 11551 | CTCCTTGATG AGCCCCAGAT ATCGACAAGT TCACAGAGAA GGGCGGACTG |
| 11601 | TCCTTCTCCC TGTTGCTGTG GGCCACGCTG AACGCCGCCT TCGTGCTCGT |
| 11651 | GGGCTCTGTG ATTGTGGCTT TCATAGAGGT GGGTGGCAGG ATGCCGCAGC |
| 11701 | TATGGCGGAC CCCATGAAGG ATTGGGGGTC TTGGTGAATG GGCGGGAACC |
| 11751 | CCTGCAGCTC ACCCACCCCC ACCATCACAT TGGCTGACAA CCCGGGCACT |
| 11801 | TTTAGAATCA CGTGGTCCAG ACTCACAACC TCAGGAGGAG CAGACACACC |
| 11851 | AGGGCCTCTT CACCCCCAGA GCCCTGGGGT GCTGCTCCTG ACCTACCAGC |
| 11901 | ACAGGCCTGG GCACCCTCAC CCCACTCCGC CCCTCCTTCC ATCTCCTCAC |
| 11951 | TCTGCCCTCC CCTCCTTCCA TCTCCACCTC CGCCTCCACC ACGTCCTTGA |
| 12001 | TCTGTGTCTG GGCTGGGAAG AGTGAGAGCA GCTACCCCAA CGACATGAGA |
| 12051 | CCCTTCCCTG GGGCCCCAAC GTGTGTGCTG CTCTTCCCTT CCCTGAGGCC |
| 12101 | CCGACGTGTG TGCTGAGCTC CCCTTCCCTG GGGCCCCGAA GTGTGTGCTG |
| 12151 | CTCTCCCCTT CCCTGAGGCC CCGACGTGTG TGCTGCTCTC CCCTTCCCTG |
| 12201 | AGGCCCCGAC ATGTGTGCTG AGCTCCCCTT CCCTGGGGCC CCGACGTGTG |
| 12251 | TGCTGAGCTC CCCTTCCCTG AGGCCCCGAC GTGTGTGCCG CTCTCCCCTT |
| 12301 | CCCTGGGGCC CCGAAGTGTG TGCTGAGCTC CCCTTCCCTG GGGCCCCGAA |
| 12351 | GTGTGTGCTG AGCTCCCCTT CCCTGAGGCC CCGACATGTG TGCTGAGCTC |
| 12401 | CCCTTCCCTG AGGCCCCGAC GTGTGTGCCG CTCTCCCCTT CCCTGGGGCC |
| 12451 | CCGAAGTGTG TGCTGAGCTC CCCTTCCCTG GGGCCCCGAA GTGTGTGCTG |
| 12501 | AGCTCCCCTT CCCTGAGGCC CCGACATGTG TGCTGCTCTC CCCTTCCCTG |
| 12551 | GGGCCCCGAA GTGTGTGCTG AGCTCCCCTT CCCTGAGGCC CCGACATGTG |
| 12601 | TGCTGCTCTC CCCTTCCCTG AGGCCCCGAC GCGTGTGCTG CTCTCCCCTT |
| 12651 | CCCTGATGCC CCGACGTGTG TGCTGAGCTC CCCTTCCCTG GGGCCCCGAC |
| 12701 | GTGTGCGCTG CTCTCCCCTT CCCTGGGGCC CTGACGTGTG TGCTGCTCTT |
| 12751 | CCCTTCCCTG GGGCCCCGAC GTTTGTGTGC TGAGCTCCCC TTCCCTGAGG |
| 12801 | CCCCGACGTG TCTGCTGCTC TCCTCAGCTC CTGGGGCTCC TGGGGCTGAG |
| 12851 | GGGCTGCATC TGTCTCAGCC TGGCCGTGAC CCACTCAGCC GTGCTTCCCC |
| 12901 | TCTTTCAGCC GGTGGCTGCT GGCAGCGGAA TCCCCCAGAT CAAGTGCTTC |
| 12951 | CTCAACGGGG TGAAGATCCC CCACGTGGTG CGGCTCAAGG TGAGGGTGCG |
| 13001 | GTGGCCCTGG CTGGGCAGGG TGGGCGCCCG CTCTTTGCTG GTTCAGGAGC |
| 13051 | AGCTCTCTTG GCTGCCTGCC TTCCAGAACT GGCCTCAGCC ACCCTGTGTA |
| 13101 | CTGGTGGCAC TGTGTGCAGA TGGGCTGGCT GGGTGTGAAG GGGTCACCTT |
| 13151 | TTTTTCTGAA AGTGGTAACA ACTGGTATTT GCACATATTA AATTACGTAA |
| 13201 | GAAATGAGTA GTCATACAGA AATGCTTGCG TGGTGCATGT GTGACACAGC |
| 13251 | TGTGCGACGC GTCTGTGACT GTGGGCTGCG TGGTGGTGAC TGATTCACCG |
| 13301 | TGGAAGCTGT CGTGGTAGTG GGCGTGTAGC AGTTTCCCGC TTTCAGTTTG |
| 13351 | CCTCATGGTC ATTTACACTT GGTGTTATCA GAGCATCTGG TTCTGGAGGT |
| 13401 | GCTGGGAGTC CTGACCCAGT TCCGCTGTGG TTGCTTCTGT CTGTGCCGCC |
| 13451 | ATCGTTCCTT AGCCTGAGAC TTGCCGCAGC CCCGTCCCGT CTGAGGATGG |
| 13501 | GTGGGCAGCA TGGCCGCTGC CCCCTGGGGG TGCTTCCGGG GCCTGGTCCC |
| 13551 | CGTGGCCAAG GAGCGGGACC AGTGTGTCCC CTCTGGCGAA AGCTCCCAGG |
| 13601 | TGACCTTGGG GTGCCCCTGC CCTGTGGTGG GAGATCAGGT TTACTGGAGC |
| 13651 | AGCTGGGAAT GGCGACCCGC CTGTCACCCG CGCCAGGCTG GCCTGAACCT |
| 13701 | TCTTGGATGT TGCTCTATAA CTTTTGTTGG CTGAGGGTTG AGTTTGCTCG |
| 13751 | GCATCTTTAA CATACAGTCC TCCCCCACAC ACTCAGCGCC CTTGTGTTTA |
| 13801 | GGGTCTGCGC CCTTGTGGGT TCTGCCCTGG GGCAGGGAGG CTGATAAACA |
| 13851 | CCTTACACAC CTTCTCAGGT GGAGAGGATG AGGCCCCTGG GGGCGGGGAG |
| 13901 | CAGCCGAAGG GAGAGGGGGC ATCGTGGAGC CGCAGGTGAC CAGCCTTCCA |
| 13951 | GTGCCAGGGG TGTATGAGGA GCCTTGCTAG GCGGGGCTAG CGGGAACACC |
| 14001 | TCCCCTGTGC TGGCCACGCT GGCGGAGGCA GGTGTGCCTG TAGGATGCGG |
| 14051 | TGGGCGGCCC AGCTTTGCCT CAGGAAGGAA GGAAACGAAA GAACCCCTTG |
| 14101 | CCTGCTCAGT GCTGAGGCCA CAGAGGGCAG GTCCCCCGAG TGAGTGCGGG |
| 14151 | GGACGCTTGG CTGCTGTTTA GCTCCACTGT GGCCATGGGG AGACCCAGCC |
| 14201 | TGGGGGTGCT GGCCCCCTCC CGGAGGCCCC GTGTCCCAGC CACTCTGCCT |
| 14251 | GATCGGGGCT GTGTGTGCTG TTTTACGGCT CAGGTCCAAA GACAGCGCCT |
| 14301 | GCCTTTTCAT CAGAGGCCAT GCGTCTCCCT GTGTTTCAGA CGTTGGTGAT |
| 14351 | CAAAGTGTCC GGTGTGATCC TGTCCGTGGT CGGGGGCCTG GCCGTGGGAA |
| 14401 | AGGTAACAAA GTGCACATGG CCACTCCAGC TGGAGCCTGA GGCCGCCGGG |
| 14451 | CCCGCGAGGG CCGCCACGCC CATGTGTGTC TCTGAGCACC GGTCCTTCTG |
| 14501 | GTCTCCAGGA AGGGCCGCGT CACGCGTGTC TCTGAGCACC GGTCCTTCTG |
| 14551 | GTCTCCAGGA AGGGCCGCGT CACGCGTGTC TCTGAGCACC GGTCCTTCTG |
| 14601 | GTCTCCAGGA AGGGCCGCGT CACGCGTGTC TCTGAGCATC GGTCCTTCTG |
| 14651 | GTCTCCAGGA AGGGCCGCGT CACGCGTGTC TCTGAGCATC GGTCCTTCTG |
| 14701 | GTCTCCAGGA AGGGCCGCGT CACGCGTGTC TCTGAGCACC GGTCCTTCTG |
| 14751 | GTCTCCAGGA AGGGCCGATG ATCCACTCAG GTTCAGTGAT TGCCGCCGGG |
| 14801 | ATCTCTCAGG GAAGGTCAAC GTCACTGAAA CGAGATTTCA AGGTGAGTTG |
| 14851 | AAATCTTGTG TGGGTGGGCT CCAGATGCCA TGGGCACGGG CACGGGCACC |
| 14901 | ACTCAGGGAG ATGGGCTTCC CTCAGCACCC CCAGGCCGAG AGCCCCAGCC |
| 14951 | CCATCTGAGG ACAGCCTGGC GGGTGGCTCC CAGAGCCAGC GGGCACAGTC |
| 15001 | CCTGCCCGGC AAGGCCTCCC TACGGCCCGC TGCTTCCCTC CTTGGGTCCC |
| 15051 | CTGCCACACG TGCATCAGTG TTTCCCGTGG GAGGGTCTGT GGCTCCAAGC |
| 15101 | GGCTTCTCAG AGGAGTGCAG AACCTGAGAC CAAGTGTGCC CACCTGTTGT |
| 15151 | TTATTTGTCA AGACACACTT TGGAACACTT TTTCCCCAAA AAAGTCCCCA |
| 15201 | GCATGTTGAT GGGGATTGAG CTGCATTTGT GTGTGATTGT ATTTTTTTTT |
| 15251 | TTTTTTTGAG ATGGAGTCTC TCTGTTGCCC AGGCTGGAGT GCAGTGGTAC |
| 15301 | AATCTCAGCT CACTGCAGCC TCCACCTCCC AGGTTCAAGC AATTCTCCTG |
| 15351 | CCTCAGCATC CCGAGTAGCT GGGATTATAG GTGCCCGCCA CCACGCCTGG |
| 15401 | CTAAGTTTTT TGTATTTTTA GTAGAGATGG GGTTTTGCCA TGTTGGCCGG |
| 15451 | GCTGGTCTCA AACTCCCGAC CTCAGGTGAT CCGCCTGCCT CGGCCTCCCA |
| 15501 | AAGTGCTGGG ATGACAGGCG TGAGGCACCG CGCCGGCCAT GTGTGAATTT |
| 15551 | AGAGGCAGGC AGCGTCCCGC AGGACAAAGA ACAGCAAGGC TGGGTTTCCA |
| 15601 | TCCGTGCGCT TTTCGTTAGA GGGTAGAGGT TTTTGGAATC TTGCGTGCGC |
| 15651 | TGGAAAGTGG AGCTCCTGGC TGGGTGTTTG CGTGTTTCCC TGGGCTGCCG |
| 15701 | GTGGTGGTGC TGACCCTGCT GTCTCTTGCC GTGGTCTGCA GCACGGTGCT |
| 15751 | CTTCAGGAAT CAGAGCTGCT GACTCGGTTG TCCTGAAAGC CCCTTCCCCT |
| 15801 | GCACAGCCCC TGTCCTGGCA GTTGCTCTCC CTTTCTGAGA GCCGTGCCCT |
| 15851 | CAAGGAACCT GCCCCGACCC TGGTCTGTCC CTGTTGCAGA TCTTCGAGTA |
| 15901 | CTTCCGCAGA GACACAGAGA AGCGGGACTT CGTCTCCGCA GGGGCTGCGG |
| 15951 | CCGGAGTGTC AGCGGCGTTT GGAGCCCCCG TGGGTGAGGA GGGCCGCACC |
| 16001 | GGGTCCAATG CTTTGCCCTC GCCCTGTGTG TTGGAAGGAA CGGTCTCCTC |
| 16051 | TCTGTAGGCC CAGTGCCCGC TGAGGGTGGC AGAGGCTTGG AGTCACGGCC |
| 16101 | GGGGCATTTG GAAGCGGCCG GCAGTGTACT TGGGTCCAGC CCTCAGACCT |
| 16151 | CCCTCAGGGT CCCTCTCTGT GTGGCTGGGG CCCACCCCAT TAGCTTCTTT |
| 16201 | CTGACGTGGT CTGGGTTCCC TGGAGCCTGG GGGAGGGAGT TGGTGGTGGG |
| 16251 | CATGGTGCCC TGTGTCCAGC TGGCACCCGA GCCGGCCGCC TGCCTTCCAG |
| 16301 | GTGGGGTCCT GTTCAGCTTG GAGGAGGGTG CGTCCTTCTG GAACCAGTTC |
| 16351 | CTGACCTGGA GGATCCTAAG TTCCTGCTGA TGGCTGCCTC CTGATCAGGG |
| 16401 | TGCATGCTGC GCTCTCATTT CCCACCATGG GGTCCACCTT GGGGCCACCC |
| 16451 | ATCGAGCTGC GGCTGGAGCT GGACCCCCTG TGGGTCTGTC AGCCCTTGGT |
| 16501 | CCTGCCCAAA GCAGCGGTCC TGCCTTTGCT GCCCAGTTCG CCCTTGGTCC |
| 16551 | TGGGCACCAT TGCCAGCCCT GGGTGGCTCC CGGGTAGGGG ATCAAACAGC |
| 16601 | CGGGAACCCA GCCCTGCCCC ACCTTCCCCT CTTGCTCTCC ACTGGCAAGT |
| 16651 | CCAGAGAGGG CTGGGCCGCT CCTTGCCCGC ACAGTGCGCC CACCCCTGGC |
| 16701 | TCCAGCCCCT TCCCTTCTGC CTTGGGCGGG GTCTGCAGAC TCCTGGCCCC |
| 16751 | GGGGCTGACA GGAGGGGCGA TGGTCCCTGC TGGTCCGTGA GCCCTGGGCT |
| 16801 | GGGAGCGTGG CTCTGAGGGC GCTGGTTTCC TGCCCTCTGC CGCAGTTCTT |
| 16851 | TGCTTCCATG ATCTCCACGT TCACCCTGAA TTTTGTTCTG AGCATTTACC |
| 16901 | ACGGGAACAT GTGGGACCTG TCCAGCCCAG GCCTCATCAA CTTCGGAAGG |
| 16951 | TTTGACTCGG AGGTAACCTG CCCCATCGCC CACCTCGCCC ACCTCGTATC |
| 17001 | CTGGTCCAGG ACCCTGTTTG CTTAAGGCCC AGGTTGAGAA TTTGGTCCTT |
| 17051 | TAGAAAACTC TGGTTGATAG CTGTGGAGCT GAGAGCTCTT GTGTAAGCTC |
| 17101 | CAGGGCCCCG AGGGGCTGCA GGAAGACACC CCAAGCTGCC CCTCAGGTCA |
| 17151 | GGGCACCATG TGACCAGCAG GGCACCTGGG ATGTCACACA GTTGCTGCGT |
| 17201 | GCATGGGGCC TCCCACGGCC TGGGGGCACG TGCAGCAGCC GCTCTCGGGG |
| 17251 | CAGGTGGGCT CAGGCCTAGT TTCCAGGGTA GCCTGGGGCC TGGGCTGGGG |
| 17301 | AGACTCTCCG TGCCATCGAT AGGGCGGCTC TGTGCGCAGG AAACTGGGGG |
| 17351 | ACCACGGGCT ATGTTCCCAG TGCTTGGGGC CCTCCCCGCC CCGGGTGCTG |
| 17401 | AGGGTGGCAG GGTCTCTGAG AGCCTCGCTG GCCACCCCGC CAGGCAGGGG |
| 17451 | CCAGGCCTGC TCAGAACACC CAGTGTGTTT CTCCCCTGTG GACTTCCGCA |
| 17501 | GCCTGCGTGG AAGGGCGGGA AGGCTCTCTG TGGGGACAGC TCTCTTAAGA |
| 17551 | TGGTGGTCCT TGAGTTTCAG CAGAAAGGAG CTGTGGGCCT TTTCCCTCAC |
| 17601 | ATCCTCTGCC TTCTCCCTCT CTCTGCACAG AAAATGGCCT ACACGATCCA |
| 17651 | CGAGATCCCG GTCTTCATCG CCATGGGCGT GGTGGGTAAG GGCTTCTCCC |
| 17701 | AGCACCGCAG GGACGGCCTG CGGGCCTGGC TCAGCTGTGA CGTGGCCATA |
| 17751 | GAGACGAGGA CTGGAGGCTG TGGCTCCCTG GAGCCTGCCC TCATCCCAGG |
| 17801 | GCCACCCGGG GGCCTCCAGA TTCTTCCATG GGCAGTACAC GTGGGGAGTG |
| 17851 | GGGAGCCCAA AGCTTCGCTT CTGTGGCTTC CCGTTGTTTA TCTCTGTTGG |
| 17901 | CAAAAACCAC AGGGCTGCAG GGATGGATGG GATTTCCTGT AAGAGATAGA |
| 17951 | ATTGCTCCCA CCAGTATTTA TTGCTCTGCT GGACACCTTT GCCCTGGAAG |
| 18001 | GAAGGCAGAG CCTTTGAGAA ACAGCTCCCC CAGCCCTCAG GGTGTGATGA |
| 18051 | TGTGGAGGAA GCATCTTACC AGGACCCCCT AGCCCCCTGC CGTCCCCTTC |
| 18101 | CCTCTGCAAA CCCTCCAGCT TCTCCTGCCA TCTGGGAGCC GGCGGGCGGA |
| 18151 | GGCCCGCACT TTTCCTCCGG TGTCGCTGAC TGGCCTTTCC CCTGTTCGCA |
| 18201 | GGCGGTGTGC TTGGAGCAGT GTTCAATGCC TTGAACTACT GGCTGACCAT |
| 18251 | GTTTCGAATC AGGTGAGGAG AAACCGCATT GCATATCGCG TTGGCAGGCG |
| 18301 | TGGCCACACA GGCCCTTTGA AAGCGGACGT GGTGGAATGG GGTTTACACT |
| 18351 | CCTAGGCCAC AGCCGAAAGA AAGGCTGTGT ATGCAGCGTC CTTCCTGATG |
| 18401 | GTTTCCCCGG TGGAGCTGGT CAGAGATGTG TCCCGGGGCC TGGAGGGTGA |
| 18451 | CGGACTAGCC CAAGGCTAGG AGTGCGAGGG CTCCTGGAGG ACGGCCCCTG |
| 18501 | GGTAGGAAGT GAGGCCCTGC GTGGGATCGG GCCTGGGCGA GGCATGCCCA |
| 18551 | ACCTTCACCG CCTGGCTCTG CCTGGTAGCA ACCGCAGCTG TCCTGGGACA |
| 18601 | CCGGGGCCCC CCGGCTTCTT CCTTCTTGGT CTGTGCTGAT TTCAATACTG |
| 18651 | TCGGGTACAG CCGGGGCACG GGTAGCGCCA CTTCCCACAC ATCTGGAGAA |
| 18701 | GTTGCTGCCG AGGAGTCTTT ACCCCAGGGA AGAGGACGAC CCCAGGACAT |
| 18751 | TTGGGTGCCT GATTGATGAT TAAACACAGG CCTGGCCGGG CGCGGTGCCT |
| 18801 | CACGACTATA ATCCCAGCAC TTTGGGAGGC CGAGGCGGGT GGATCACCTG |
| 18851 | AGGTCGGGAG TTCTAGACCA GCTTGACCAA CATGGAGAAA CCCCGTCTCT |
| 18901 | ACTAAAAAAT TCAAAAAAAA ATTAGCCAGA TGTAGAGCCG GGCGCCTGTA |
| 18951 | ATCCCAGCTA CTCGGGAGGC TGAGGCAAGA CAATTGCTTG AACCTGGGAG |
| 19001 | GTGGAGGTTG CAGTGAGCCA AGATCGCAGC ACTGCACTCC AGCCTGGGCA |
| 19051 | ACAAGAGCAA AACTCCGTCT CAAAAACAAA AACAAACAAA CAAAAAGCAC |
| 19101 | CACGGGCCCA GTGTCCTCCA TCAGGGACTC GAGTTGCCAT GGGGCCTGCG |
| 19151 | GAGGGGCCGC GCTGCCGTCC TGCCTGCCAT GCAGCCTGAT TCTTGGTTCC |
| 19201 | AGGTACATCC ACCGGCCCTG CCTGCAGGTG ATTGAGGCCG TGCTGGTGGC |
| 19251 | CGCCGTCACG GCCACAGTTG CCTTCGTGCT GATCTACTCG TCGCGGGATT |
| 19301 | GCCAGCCCCT GCAGGGGGGC TCCATGTCCT ACCCGCTGCA GGTGGGAGGC |
| 19351 | TGGGCCCGGG CGGGGTCCAG CAGGCAGGGC AGCCACAGGG CGGCCTCCAG |
| 19401 | GAGGCTCGCT TAGGCTACGG GAGGAGGGCT GCCCACCCCG CCGAGTTCCA |
| 19451 | GAAGCGCATG GGCTGGCGTG TCTCAAAGAG GGTTAGTCCT GTCCACCCAG |
| 19501 | ATCTCAGAGG AGGCCAGGTG TCTGCTGAGG TGCCAGGGGA ATGGGCGGTG |
| 19551 | GTATGGGGGC CAGAGGCTCC CCCCAGTCCT CTTCCCAGAA TGGCAGCCTG |
| 19601 | ACGGGGCGAG CCTCAGGCGC CCTATGGGGG CACCATAGAT GTGGACCCAG |
| 19651 | GAGAAATGCA AACCTCCGTC CACAACTGGA CCTGTGCCTG GCGCTCACGG |
| 19701 | CTCACCGCCG TCCGTGCGTC CATCTGCACT GTGACACGGT TGCCCTGGAA |
| 19751 | AGCACTACGC TCAGAGGAAC CACACGTGAG GTCACGCGAC GTAGCCCCAT |
| 19801 | TAACATGAAA CATCCAGAAC AGGGAGAGCC TAGAGGCCCA GCAGACCAGT |
| 19851 | GGGTGCCACG GCGGGAGTGG GCAGGATGGG ACGGGTCAGG TGTGAACCGT |
| 19901 | TAGAGACGTG GGAGGCCCGG GGCCATGGGG TTGACCAGCC TTGCTACACT |
| 19951 | CTGCTCCAGC CCCGTGGATA ACACCCCCTG TGCTGCTGGA GCCCAGGAGG |
| 20001 | CTCTGGGCCT GTGGCACCGG GGCGCCAACA GCCTCTTCTA GGAGCTCATG |
| 20051 | TGAGCGCCTG GGCCCACCTT CCCCGGCACC AGGGATGGAC AGCGTCTCAG |
| 20101 | CCCATGGTCC TGCTAACCCA CCCCCCAGGG CTAGACACGG CCCCCTGCTG |
| 20151 | GGCCTAGGCC GTGTGTGTCC TCCTTTCCCT CCGTGACCAT GGCTTGGGCC |
| 20201 | TTGTGTGTCC TCCTTGCCCT CTGTGACCGT GGCCCTGACC CAATGGCAGG |
| 20251 | ATCGTGTGGT TTCGCGCCTG ATGCTGGCCA GGCACAGGGT ACACGGCCTC |
| 20301 | TCACGGCGAC ACCAGGTTTG TGCCTGCAGC CCACCAGCTC ATCTCCCCTC |
| 20351 | CCAACGTGTG CTCTCTCCCG ACCCCACAGC TCTTTTGTGC AGATGGCGAG |
| 20401 | TACAACTCCA TGGCTGCGGC CTTCTTCAAC ACCCCGGAGA AGAGCGTGGT |
| 20451 | GAGCCTCTTC CACGACCCGC CAGGTGTGTG TGGGCAGTGC CGCTGGGCAG |
| 20501 | GCCCTGGGAT CAGGGCCTGG GTGATGCCTT CTGGCTGAGT GTCCCCTGTG |
| 20551 | GGCTGAGGTT GCAGCCCTGG GCTGGGGGGT CATCCCTAGC ATGGATCATA |
| 20601 | GCAGGGACTC ACTCCTGTAA TCCCAGCACT TGGAGAGACC AAGGCAGGAG |
| 20651 | GATCACTTGA GCCTAGGAGG TTAAGACCAG CCTGGGCAAC TTAGCGAGAC |
| 20701 | TCTGTCTTTG CAAAAAAGCA ACATTATCTG GCTACGGTAG TACACCCACA |
| 20751 | GTCCCAGGTA CTTGGGAGGC TGGGCCGGGA GGATTGCTTG AGCCCAGAAG |
| 20801 | GTTGAGGCCA CAATGAGCTG TGATTACATC ACTGCATACC AGCCTGGGTG |
| 20851 | ACACAGCGAG ACCCTCTCTC AAAAAACAAA AGAAAACCCA GCCTGGTGAC |
| 20901 | TCCCACACCA AGACCACGGC CTGGCCTCGC TCGACCACAA GTGTTTCACG |
| 20951 | GAAGCGCAGA CCGCGACCTT GGAGTGCCGG CCTTTCACCT CTGCAGTTGT |
| 21001 | GTCCCTGGCG GTCTCACCCG CCCTGCACGC AGTACAGTGC TGCCTGCTCC |
| 21051 | AGGAAAGGAA CCCCAGGCTG TGGCGGGCAC CCTCTTCCCG GAGCCAGGCT |
| 21101 | GCGAGCTGCA CCACGGTGCA CACCCATGGA GTGTAGACCT GGCGCTGCTA |
| 21151 | GACCCAGCTC GGCCGCCCCG CTGGACGCGG CTCCTGCTTC TGCTGGCATC |
| 21201 | AGGGCCCCGC AGAGCCTCTT CCCCTGTGGC CTCCCCATGG GATCCTTTTA |
| 21251 | GCCTTTCTGC TTCCCAGGGA GGCTGAGAAC AGGGAGCCTT CTGGGGACCG |
| 21301 | CTGGGCTCGG GAGCTCAGGT TGCTGGGCTC CTGGAAGGTG ACTGTGAGGC |
| 21351 | CCGAGACTGG GCAGCGGGGC AGGGCAGTCC TGCGGAGGCG GGAGTCGTGG |
| 21401 | AGGCCCCGTC AGCCCCTCTT CTCTCCTAGG CTCCTACAAC CCCCTGACCC |
| 21451 | TCGGCCTGTT CACGCTGGTC TACTTCTTCC TGGCCTGCTG GACCTACGGG |
| 21501 | CTCACGGTGT CTGCCGGGGT CTTCATCCCG TCCCTGCTCA TCGGGGCTGC |
| 21551 | CTGGGGCCGG CTCTTTGGGA TCTCCCTGTC CTACCTCACG GGGGCGGCGG |
| 21601 | TGAGTGGGGC CGGAGGGGAG GCTGTGGGGC CCTGCAGCTG AGCCAGGTCT |
| 21651 | TGCGGCATCG CGGGCCGGAG CAGAAGTCCC AGGGCAGGAC AAAAGTGTCG |
| 21701 | CACCTCACGT GGTTCACGGG CCGTGGGCGT TGTCCTCGCG TGGTTCACGG |
| 21751 | GCCGTGGGCG TTGTCCTGCT GTGGTGGCAG CGTGTACTGT GGCAGCGCAG |
| 21801 | CCCATGTGTG GAGTCTGGAC CAGGCGAAGG TAGGGGGCGG AGGCTCGTGT |
| 21851 | CCTTATTCTT GAGAATGTGA TGAAAAGCAG AGGTGATTGT GGGCTGCTGC |
| 21901 | AGAGCTGTTT CTAGACTCCA TGGGGTGGAT GTCCGGCCAG GGCTGCTCTC |
| 21951 | TGTGAGGCCG GGGGCCAGAG CGGCATACAC TGCCCTCCAG ACCTCAGCCC |
| 22001 | CCGCAGGCCT TCCTTCTCTG CCTGCCTCTG CTGGGACTGG GTTCTCTTAT |
| 22051 | GTGTCTTCTG TTTCTCATTT CAGTCGCTTA AATAAGACTG AAAACCTGTA |
| 22101 | AGAGGCCCTG GCAGGAAGCC CCCGGCCATG CTTCTCATCC CCGGCAGGAA |
| 22151 | GCGCCCACTC CTGCTCCCCA GGCCCGTGTG CTCTGCCCAT CTCCCTCCGC |
| 22201 | ACAAGGGTTT GGTTTGGTTT TTAAAATTGA AACATGATTC AAATACCGTA |
| 22251 | AAACTCATCG TTTTAAAGAG GGCAGTTCAG CGGCGTTTCT CACGTTCACG |
| 22301 | AGGCAGTGCG GCCGTCACTA CCACTTCTAG AATGTTCCGT CATCCCAGAA |
| 22351 | TGGAAACCCT GTGCCCACCG ACCCTCGTGC CCCGCTTTCT GCAGCCTCCA |
| 22401 | TGCCTGGGTT CTGTGGCCCA GCCTGATGTT CCCGGGGCTC TCTGTGTCGT |
| 22451 | GTGTGCCGGG GTTTCACTCC TCATGCTGGA CGGTGCTCCC TAGTTGGCCT |
| 22501 | GGGCTGCTGC GTGGTGACTG TGCCCTCTGC ATCCTCCATG CCTGCCACTC |
| 22551 | CCCTGTTGCT CGGGTGCTGA GCGCCTGGTT CAGGCCAAGG ATGCAGCCTC |
| 22601 | CGCAGCAGGG TGTACTGTGC TAGGTTGTTC TGTGTGTATG TACGCGGCCA |
| 22651 | CGAGGTTTGT TCCTGGCTGT GGGGCTGCTG GGCCTGGGCA GGGCCTCCTC |
| 22701 | CGTCTGTGTA TCTTGGTGGG TTTGGGCCTG CCACCACACT GACACCTCCT |
| 22751 | CCGTGTCACC TCCCACAGAT CTGGGCGGAC CCCGGCAAAT ACGCCCTGAT |
| 22801 | GGGAGCTGCT GCCCAGCTGG GTATGTCCCA GCTCTTGCCC GATGGGTGGG |
| 22851 | GAGCTCCACG GGGTCTGGAG GGGGCCATGG CTGTCCTTGC GGGGCTAGGG |
| 22901 | TCTGGGAGCA GGTGGATGGG ATGGGTGCTG CAGAGAAGGC AGTGGCCACG |
| 22951 | TGACCCTGAG CCAGGAGGGT GGACGTGCTG GGGTTCATGA TGGCTCCCGC |
| 23001 | AGGCGGGATT GTGCGGATGA CACTGAGCCT GACCGTCATC ATGATGGAGG |
| 23051 | CCACCAGCAA CGTGACCTAC GGCTTCCCCA TCATGCTGGT GCTCATGACC |
| 23101 | GCCAAGATCG TGGGCGACGT CTTCATTGAG GTGCGCCAGG GCCTCGAAGC |
| 23151 | CTCACCCTGA GAGCGTGGGT GCTGCCATAG GGGAGGGGCC CCTGTGAGCC |
| 23201 | TCCAAACAGC CGGTCCCGGG GGGTAGGCTC AGGGTTTCTG GGGGCGGCCT |
| 23251 | CTGGGCTCCC AGGGGTAGGC TCGGGGCTCC AGGGGTGGGT GTGGACTCCT |
| 23301 | CAAGCCCTGT GTTCCCGCCC CGCCCGCAGG GCCTGTACGA CATGCACATT |
| 23351 | CAGCTGCAGA GTGTGCCCTT CCTGCACTGG GAGGCCCCGG TCACCTCACA |
| 23401 | CTCACTCACT GCCAGGTACA GCGCCCAGGA CACCTGTGGG TGGGGAGGGT |
| 23451 | GTCCAGCGGC CTCTTGTTGC ACAGGGGCAG GGTGCACGGC TTGCGGGCTC |
| 23501 | CAGGCAGCCC CGCGTTTCCT GTCCAGCGGC TTCACACCTC ACCAGCCCGC |
| 23551 | AGAGGTAACT GTGGGAGTTG GTGGCGTGTG ACGGGCATGT GTGGCCGGGC |
| 23601 | TCCTCCGGCA GGGAGGTGAT GAGCACACCA GTGACCTGCC TGAGGCGGCG |
| 23651 | TGAGAAGGTC GGCGTCATTG TGGACGTGCT GAGCGACACG GCGTCCAATC |
| 23701 | ACAACGGCTT CCCCGTGGTG GAGCATGCCG ATGACACCCA GGTACCGGGC |
| 23751 | ACCCCATAGA CAGGGTCCTG CCTATGTGAC CTCTGTCGAG TCCATTGGTG |
| 23801 | GGAAGCACAC GGCAAGGTTT GCAGGATGGG GAAGCTGCAC GTTTGGGTGC |
| 23851 | ACTGGCAGTT CCAGGAGTGC CGGAAGCTGA GTGTGCAGCC ATGGAGGGCT |
| 23901 | GTGTGGACGC TGAGGCTGGT GGGGGGGGCT GCGGCCTGGC AGGGTCTTGG |
| 23951 | GGTTGGCACC CAGGCTGGGC TGAGAGCCGT GGCACTGGGG GCCGTGACTT |
| 24001 | TGTCAGGAGG CCCTGACAGG ACACACAGCT CGGCCACTGC TGTGTGTCTT |
| 24051 | TTAGACGTGG ACACTGGGTG TTTGGAGGTT GGTTTTTATT GGGACCCAGT |
| 24101 | GGGGCTGCAT CTGCCCTGCA GCAAAGCCAC CATCCCTGGG CCCTTGGCTC |
| 24151 | TCTGCTGTGC GCGGTCAGGC CCCGCTACCC TGTCGCCGAT CCTTGGGTCC |
| 24201 | CGTGGCATTG TGCGTGTGGG ATGCCATGGC GAGGCTGGTG TGAGCAGGTA |
| 24251 | GCCACCGACA CGGGGCCCAT GCCCAGATGG GAAATCTGGC CGGAACAGGG |
| 24301 | TCAGAGCGGG GCCCGACACA GCATTCCAGC GCAGCCTCCC ACCCTCGGGC |
| 24351 | CCGTGGCCCT GACCGCGGGC CTGTCTTGCA GCCTGCCCGG CTCCAGGGCC |
| 24401 | TGATCCTGCG CTCCCAGCTC ATCGTTCTCC TAAAGCACAA GGTGCGTGCC |
| 24451 | AGGCTCCGGG CCATTGGGCG GGTGGGGGCC CCGGGGGTGC TGCCTGGGTG |
| 24501 | CCTGACACAG GGCTCTGCCG CCCGCAGGTG TTTGTGGAGC GGTCCAACCT |
| 24551 | GGGCCTGGTA CAGCGGCGCC TGAGGCTGAA GGACTTCCGA GACGCCTACC |
| 24601 | CGCGCTTCCC ACCCATCCAG TCCATCCACG TGTCCCAGGA CGAGCGGGAG |
| 24651 | TGCACCATGG ACCTCTCCGA GTTCATGAAC CCCTCCCCCT ACACGGTGCC |
| 24701 | CCAGGCATGT GCAGGGCATG GGCATGGGCG TGGGGCCTGG GACTGAACAG |
| 24751 | CAGGGGGTGG GGTCCAGAGC CTCGGGGAGG GGCAGCCGGG GGGGGCCACA |
| 24801 | GCGGAGAGGA CTCGGTGACT CTGTCTCCTG TGAAGGGCCT GGCAGGCTTT |
| 24851 | AGAGCTGAAG TCAAGGGGCT GAGGGGGCTG GCCAGACGGG CGTGGGGGCT |
| 24901 | CAGGACGTGC CTGGACGCCG TGGTGGGGGG TGCAGGGAGC CAGCTTGGGT |
| 24951 | GAGGGTCCCG CCTGCCTCTG CTGTGTGGGC GGGCACTGAC AGCTGTGCCC |
| 25001 | CTGCTGCAGG AGGCGTCGCT CCCACGGGTG TTCAAGCTGT TCCGGGCCCT |
| 25051 | GGGCCTGCGG CACCTGGTGG TGGTGGACAA CCGCAATCAG GTGAGCGGGG |

1. A method for assessing bone mineral density (BMD) in an individual, the method comprising using a chloride channel 7 (Clcn7) gene marker.

2. A method as claimed in claim 1 for assessing lumbar spine BMD or femoral neck BMD.

3. A method as claimed in claim 1 for assessing whether the individual is at risk of a low-BMD-associated disorder.

4. A method as claimed in claim 3 for assessing whether the individual is at risk of osteoporosis or an osteoporotic fracture.

5. A method as claimed in claim 4 wherein the method comprises:
(i) obtaining a sample of nucleic acid from an individual, and
(ii) assessing a polymorphic marker in the Clcn7 sequence of the nucleic acid.

6. A method as claimed in claim 5 wherein the nucleic acid is genomic DNA.

7. A method as claimed in claim 5 wherein the polymorphic marker is a single nucleotide polymorphism (SNP) and the identity of the nucleotide at the SNP is assessed.

8. A method as claimed in claim 5 wherein the SNP is selected from the group consisting of the following positions:
(i) 19233, situated in exon 15 (Appendix 2)
(ii) 19240, situated in exon 15 (Appendix 2)
(iii) 39699 situated in exon 1 (Appendix 1)
(iv) 39705 situated in exon 1 (Appendix 1)
or a polymorphic marker which is in linkage disequilibrium with any of these.

9. A method as claimed in claim 8 wherein the identity of the nucleotide at the SNP is shown in Table 2.

10. A method as claimed in claim 9 wherein the SNP is selected from the G19240A and T19233C polymorphisms in exon 15 of the Clcn7 gene.

11. A method as claimed in claim 10 wherein:
an individual who is G/G homozygous for SNP19240 is classified as being at the lowest risk; an individual who is G/A heterozygous is classified as having moderate risk;
an individual who is A/A homozygous is classified as having lowest risk, of susceptibility to a disorder which is associated with a low BMD,

12. A method as claimed in claim 10 wherein:
an individual who is T/T homozygous for SNP19233 is classified as being at the lowest risk; an individual who is T/C heterozygous is classified as having moderate risk; an individual who is C/C homozygous is classified as having lowest risk, of susceptibility to a disorder which is associated with a low BMD.

13. A method as claimed in claim 5 wherein the polymorphic marker is a tandem repeat marker.

14. A method as claimed in claim 5, wherein the tandem repeat marker is the 50 bp repeat polymorphism at position 14476 situated in intron 8 (Appendix 2) or a polymorphic marker which is in linkage disequilibrium with this.

15. A method as claimed in claim 14 wherein the 50 bp repeat polymorphism at position 14476 situated in intron 8 (Appendix 2) is assessed and an individual carrying one or two alleles with 3 tandem repeats is classified as having a low risk of susceptibility to a disorder which is associated with low BMD.

16. A method as claimed in claim 8 wherein two or more of said Clcn7 markers are assessed.

17. A method as claimed in claim 5 wherein the Clcn7 sequence in assessed by determining the binding of an oligonucleotide probe to the nucleic acid sample, wherein the probe comprises all or part of (i) the Clcn7 genomic sequence of Appendix 1 or 2, or (ii) a polymorphic form of the Clcn7 genomic sequence shown in Appendix 1 or 2, or (iii) the complement of either.

18. A method as claimed in claim 17 wherein the probe comprise a nucleic acid sequence which binds under stringent conditions specifically to one particular allele of the Clcn7 polymorphic marker and does not bind specifically to another allele of the Clcn7 polymorphic marker.

19. A method as claimed in claim 18 wherein the probe is labelled and binding of the probe is determined by presence of the label.

20. A method as claimed in claim 5 wherein the method comprises amplifying a region of the Clcn7 sequence comprising at least one polymorphic marker.

21. A method as claimed in claim 20 wherein a region of the Clcn7 sequence is amplified by use of two oligonucleotide primers.

22. A method as claimed in claim 21 wherein at least one of said primers binds under stringent conditions specifically to one particular allele of the Clcn7 polymorphic marker and does not bind specifically to another alleles of the Clcn7 polymorphic marker.

23. A method as claimed in claim 21 wherein at least one of said primers is a mutagenic primer which introduces a restriction site into said amplified region of the Clcn7 sequence.

24. A method as claimed in claim 21 wherein at least one of said primers is a primer shown in Table 4.

25. A method as claimed in claim 5 wherein the Clcn7 sequence is assessed by a method selected from the group consisting of: strand conformation polymorphic marker analysis; heteroduplex analysis; RFLP analysis.

26. A method as claimed in claim 5 wherein the polymorphic marker is assessed or confirmed by nucleotide sequencing,

27. A method of determining the presence or absence in a test sample of a polymorphic marker in the Clcn7 sequence which is selected from the group consisting of the following positions:
(i) 14476 situated in intron 8 (Appendix 2)
(ii) 19233, situated in exon 15 (Appendix 2)
(iii) 19240, situated in exon 15 (Appendix 2)
(iv) 39699 situated in exon 1 (Appendix 1)
(v) 39705 situated in exon 1 (Appendix 1)
which method comprises determining the binding of an oligonucleotide probe to the nucleic acid sample, wherein the probe comprises all or part of (i) the Clcn7 genomic sequence of Appendix 1 or 2, or (ii) a polymorphic form of the Clcn7 genomic sequence shown in Appendix 1 or 2, or (iii) the complement of either.

28. A method of determining the presence or absence in a test sample of a polymorphic marker in the Clcn7 sequence which is selected from the group consisting of the following positions:
(i) 14476 situated in intron 8 (Appendix 2)
(ii) 19233, situated in exon 15 (Appendix 2)
(iii) 19240, situated in exon 15 (Appendix 2)
(iv) 39699 situated in exon 1 (Appendix 1)
(v) 39705 situated in exon 1 (Appendix 1)
which method comprises use of two oligonucleotide primers capable of amplifying a portion of the Clcn7 sequence which portion comprises at least one of said markers.

29. A method for mapping polymorphic markers which are associated with a disorder which is associated with a low level of bone mineral density (BMD), the method comprising identifying polymorphic markers which are in linkage disequilibrium with a marker which is selected from the group consisting of the following positions:
(i) 14476 situated in intron 8 (Appendix 2)
(ii) 19233, situated in exon 15 (Appendix 2)
(iii) 19240, situated in exon 15 (Appendix 2)
(iv) 39699 situated in exon 1 (Appendix 1)
(v) 39705 situated in exon 1 (Appendix 1).

30. An oligonucleotide probe for use in a method of claim 17

31. An oligonucleotide probe as claimed in claim 30 which comprises a Clcn7 polymorphic marker selected from the group consisting of the following positions:
(i) 14476 situated in intron 8 (Appendix 2)
(ii) 19233, situated in exon 15 (Appendix 2)
(iii) 19240, situated in exon 15 (Appendix 2)
(iv) 39699 situated in exon 1 (Appendix 1)
(v) 39705 situated in exon 1 (Appendix 1).

32. An oligonucleotide probe as claimed in claim 30 which comprises a label.

33. A PCR primer pair for use in a method of claim 20 which primer pair comprises first and second primers which hybridise to DNA in regions or including flanking the Clcn7 polymorphic marker.

34. A PCR primer pair as claimed in claim 33 wherein the Clcn7 polymorphic marker is selected from the group consisting of the following positions:
(i) 14476 situated in intron 8 (Appendix 2)
(ii) 19233, situated in exon 15 (Appendix 2)
(iii) 19240, situated in exon 15 (Appendix 2)
(iv) 39699 situated in exon 1 (Appendix 1)
(v) 39705 situated in exon 1 (Appendix 1).

35. A PCR primer pair as claimed in claim 34 wherein at least one primer is selected from Table 4.

36. A kit comprising a probe and\or primer of claim 30

37. A method of osteoporosis therapy, which method includes the step of screening an individual for a genetic predisposition to osteoporosis in accordance with the method of claim 4, whereby the predisposition is correlated with a Clcn7 polymorphic marker, and if a predisposition is identified, treating that individual to prevent or reduce the onset of osteoporosis.

38. A method as claimed in claim 37 wherein said treatment comprises hormone replacement therapy.


SEQUENCE LISTING (37 sequences)

SEQ ID NO: 1 - DNA, 350 bp, Homo sapiens
cagccggcgcttcccggccggtgtcgctccgcggcgggccatggccaacgtctctaagaaggtgtcctggtccggccgggaccgggacgacgaggaggcggcgccgctgctgcggaggacggcgcggcccggcggggggacgccgctgctgaacggggctgggcctggggctgcgcgccaggtgaggccgggcagggcgcaggcggggaaactgagccctcgtgcgccccgcagcccgcgccctcgtgagccttctggcggcgccgcgtgtctcggtcctggaggcgaccgaggcgcggtggactcgggaacgcgccccggggctcctcggcggggccgggctggcgggg

SEQ ID NO: 2 - DNA, 18700 bp, Homo sapiens
aggatggcccagggtgctgtggcgggcactgcattgggggcggcgtgttgtccagcccttctttcctggtgggtggcaggtgcctcgctttcagtctagagcagggagcttgcgccctggactcgggctggacgtgtcgctgacaggccgaggggcagccggatcagttctgcttccagggcccagggaggcccgtcccagccctgctgcccccacccagcaggcaggcctggcctagcccattcctgagctcccgggcagggtcaggcgaggccagggtgcggcggcgggagtgagaatccacggagcagagcgtgcgacgcctgagcgccctcatgatttctcttctgcttttagtcaccacgttctgcgcttttccgagtcggacatatgagcagcgtggagctggatgatgaacttttggacccggtgagttgggggtgttccccgtcctcccgcagagctagctgcatcttagcagagggtgacagggatgggcacgggccgagcggcagggagatagtggggccccagggccggggttcagggaagatttccttggggggacatggtccctgacgccaagtgagcagaggcagctgggcagaagtgctctcagacggaggagtgcagggcgcaggaagccggtcaggacagcagtgacagcatgggcagcgagggggctggacctggctttgggacagggcaaggacagggatcttgggggggcagtgaggagccccaggagagtgagagggggccggatgcctctgacttcagagggcaggggtttagatgttcccgtgccagtggctgccctgggagtcctgagctcagcggcagcgtgctcgtcttccttcccctcgggggcatctcccgccggcctcggtttttcccccagccgctggtgaggccgggagtcctctgctgccgctggccgttcactcatcgtctctgggtagatgtctgtgcgggactcctgttgagatgatcctgatgttggcaacaccccgggcgtcctccttctccccatcaggccccacctggctctgccctgggccacgtcagaggctgaggcatctcacagtccacctgtccgggtgctcttcggccttgcgtccgtttgagctctgccgcagtcgctcccgaggccggcgccgtgctcagatgccgtcctgtacagccagcagcgcctcttccggggctgcccttctgatacgtttgtgctgcctctggagccacaaggccttcggaagatctgtttcgtggccgtgggcgccttcggcactgcctttttggacttcaaagcctttgctctggtgtcagctttgggaggggcaggagttgggagagaagggaaaaagccagcacgtgagattcagcaatcagtcctctcctgtctcaaccctggagcgggtgcctggccggccacacgcgtgttggttatgctcatttttaaactgggtttgttgtctttataattgagctgcaggagttctttatacatagatgcaaatctctcatccaatacatgatttatagaagttttctcccgttcagtgggttttctgttcactttctcagtggtgtcttttgttgctcaaatttatttaattaaaaaagttttggccaagggaggtgattcgtgcctgtaatcctagtactttgggaagcagatggattcattgagctcaggagttcaagatcagcctgatcaacatggtgaaaccctgtctctacaaaaaatataaatattagctgggcctggtgataggcaccagtagtcccagctacttgggaggctgaggttggaggatcacttgagcccaggaggtggaggtttcagtgagctgagatggtgccactgcacttcagcctgggtgacagagtgagatcctgcctcaaatttttttttttttttctgggcaggtgtggtggttcacacctgtaatcccaacactttgggaaaccaaggctgcagcccaggatttggagatcagcctagacaacacagtgagaccctgtctctacaaaaaacaaaaacaaaaacgaaaattagccaggtgtggtggtgtgcgcctgtggtcccagctactcaggacgctgaggcaggtggattgatcgaacccaggaggttgaggctgcagtgagccatgatcacaccattgtacttcagcctgcgtgacagacgggaccctgtctaaaaaaattaattattactattctttgagatgaggtctcactgtgtggcccaggctgaactccatctctcaggctcaagcaatcctcccgtttcagcttcttcctgaggagctgggaccacaggtgcatcacaccccgcacaggttgtattgctgaggttcagctaatctgttttttcttgtgttgcttgtacttttggtgtcaaatctaagaaaccattgcctcacccaagagtatgacgactgacccgttttttcctaagaattttacagttttaggtctttcatcccttttgagttaatttttggatgtggtgtgaggtaagggtccaacgtcataccctccctctctctctctctttttttgagacagggtctcactgtcacccaggctggagtgcagtggtgcaatcatggttcactgcagcctctgcctcctgtctgtctcccaagtagctgggactcaggcgcatgtcaccatactcagctaatattttgtagagatggagtcttactatgttgcccaggctgatcacaaactcctggcctcaagcagtccttctgcctctgcctcccagagtgctgggattatagctgtcagccattgcgcccggcccagcttcatttttgcatgtggaaatccagttgtaccagcaccatttgttgaaaacactacctttctctgttgaaatgttttgacactgttgtgggaaatcaattgatcgtacatgttttggatttctttctggactctctcaattctcttccattcttttgtggccatcttcatgccagtaccatgcctggttttttttttttttttttttttggcttttttttaagagttggggtctcactgtgttgcccaggctgggtggatcacttgaggccaagagtttgagaccagcctggccaacatggtgaaaccccgtctctactaaagatacaaaaattagccaggcgtggtggtgcacacctgtaatcccagctacttgggaggctgaggcaggagaatggctttaacctggaaggcggaggttgcagtgagttgagatcgcgtcactgcactctagcctgggcaaaaagagtgactgtatctcaaaaaaaaaaaaaaaaaaaaaaaaaaaaagacagatgagggttttactctgttgcccaggctggtcttgaactcctggcttcagttgatcctcttgcctctgcctcccagagtgctgggattacaggtgtgagccaccgcacccggcctcatggattgatttttggatgttaaactaacttgtattcctaggctgaattcaccttgctcctggcattgctggaatcactttgcttgtgtcttaccaaagatctttgcatccgtggttgtaggggtgttggtctgtagttctcttttttttttttttctttgagacggagtcttgctctgtcacccaggctggagtgcaacggcgcaatctcggctcactgcaacctctgcatcccgggttcaagcgattctcctgcctcagcctcctgagtagctgggattacaggcgcccaccaccacgcccagctaatttttgtatttttagtagcgacagggtttcatcttgttgtccaggctggtctcgaactcctgaccgcagctggtccacttgcctcggcctcccaaagtgctgggattgtaggtgtcagccaccgcgccccatgtgcagttctcttgctgtgtccttgtccttggtgtcgggatgataatggcctcgtgtgtgagctgagaggggcctctctccttgtggccttgtcaactgtgcttctctctttgcctttttctgccacaggatatggaccctccacatcccttccccaaggagatcccacacaacgagaagctcctgtccctcaagtatgaggtgggcgtccttctgtccccctgaccctgagacccggcctctgccccctgccagcccactcccggtcccctgtgcccgcacccagagcgtgggttcggtgctgagtgctgcccttgctgtcccggcctgcagagcttggactatgacaacagtgagaaccagctgttcctggaggaggagcggcggatcaatcacacggtgagctggacgccgctccctgcagggccccacggtgcggggcctggtgccggccgggcctggggctgctcttctgccggggtgaggtgacgcacctcctccctcctcctctgactccgcctctgaggcctgtggttcgtctggtttctagagacagtgggagggtcacggtcaccgtaaccaagaaggctgctcttacggccgccagatgcggtgcccagcataacaaccgctggctgtgaagttgttgggaattcacccacctccccgagtcaccctcgggccccgggtgcgcctcagatgttggccagaaactgtcctttgtgggactcagcgcaccgtgcacactgggcccttcataatcccggggcctgcaggcggtctgggcggtcctgctgctgccagagtgactgcgccagggccctgcctgaccctcgccctgaccgcgccctgcaggccttccggacggtggagatcaagcgctgggtcatctgcgccctcattgggatcctcacgggcctcgtggcctgcttcattgacatcgtggtggaaaacctggctggcctcaagtacagggtcatcaagggcagtatccttcccagtgcggccgctgcagcttgggaggggggcgtggcctgggccgagtcccgggcagaagtcctgagcccagcgtgttccagtgcaggtggaggcggcccggccaggctggctgtgtccctgtcatggttgggccgtgagacgtctctgggatgtccagtgaacatcatggctccacccagcagggtggcatctgccaggctggtctgtggggcagggctgaggtctgggctgggtggtcatgacggggaagcagccagccctccttgatgagccccagatatcgacaagttcacagagaagggcggactgtccttctccctgttgctgtgggccacgctgaacgccgccttcgtgctcgtgggctctgtgattgtggctttcatagaggtgggtggcaggatgccgcagctatggcggaccccatgaaggattgggggtcttggtgaatgggcgggaacccctgcagctcacccacccccaccatcacattggctgacaacccgggcacttttagaatcacgtggtccagactcacaacctcaggaggagcagacacaccagggcctcttcacccccagagccctggggtgctgctcctgacctaccagcacaggcctgggcaccctcaccccactccgcccctccttccatctcctcactctgccctcccctccttccatctccacctccgcctccaccacgtccttgatctgtgtctgggctgggaagagtgagagcagctaccccaacgacatgagacccttccctggggccccaacgtgtgtgctgctcttcccttccctgaggccccgacgtgtgtgctgagctccccttccctggggccccgaagtgtgtgctgctctccccttccctgaggccccgacgtgtgtgctgctctccccttccctgaggccccgacatgtgtgctgagctccccttccctggggccccgacgtgtgtgctgagctccccttccctgaggccccgacgtgtgtgccgctctccccttccctggggccccgaagtgtgtgctgagctccccttccctggggccccgaagtgtgtgctgagctccccttccctgaggccccgacatgtgtgctgagctccccttccctgaggccccgacgtgtgtgccgctctccccttccctggggccccgaagtgtgtgctgagctccccttccctggggccccgaagtgtgtgctgagctccccttccctgaggccccgacatgtgtgctgctctccccttccctggggccccgaagtgtgtgctgagctccccttccctgaggccccgacatgtgtgctgctctccccttccctgaggccccgacgcgtgtgctgctctccccttccctgatgccccgacgtgtgtgctgagctccccttccctggggccccgacgtgtgcgctgctctccccttccctggggccctgacgtgtgtgctgctcttcccttccctggggccccgacgtttgtgtgctgagctccccttccctgaggccccgacgtgtctgctgctctcctcagctcctggggctcctggggctgaggggctgcatctgtctcagcctggccgtgacccactcagccgtgcttcccctctttcagccggtggctgctggcagcggaatcccccagatcaagtgcttcctcaacggggtgaagatcccccacgtggtgcggctcaaggtgagggtgcggtggccctggctgggcagggtgggcgcccgctctttgctggttcaggagcagctctcttggctgcctgccttccagaactggcctcagccaccctgtgtactggtggcactgtgtgcagatgggctggctgggtgtgaaggggtcacctttttttctgaaagtggtaacaactggtatttgcacatattaaattacgtaagaaatgagtagtcatacagaaatgcttgcgtggtgcatgtgtgacacagctgtgcgacgcgtctgtgactgtgggctgcgtggtggtgactgattcaccgtggaagctgtcgtggtagtgggcgtgtagcagtttcccgctttcagtttgcctcatggtcatttacacttggtgttatcagagcatctggttctggaggtgctgggagtcctgacccagttccgctgtggttgcttctgtctgtgccgccatcgttccttagcctgagacttgccgcagccccgtcccgtctgaggatgggtgggcagcatggccgctgccccctgggggtgcttccggggcctggtccccgtggccaaggagcgggaccagtgtgtcccctctggcgaaagctcccaggtgaccttggggtgcccctgccctgtggtgggagatcaggtttactggagcagctgggaatggcgacccgcctgtcacccgcgccaggctggcctgaaccttcttggatgttgctctataacttttgttggctgagggttgagtttgctcggcatctttaacatacagtcctcccccacacactcagcgcccttgtgtttagggtctgcgcccttgtgggttctgccctggggcagggaggctgataaacaccttacacaccttctcaggtggagaggatgaggcccctgggggcggggagcagccgaagggagagggggcatcgtggagccgcaggtgaccagccttccagtgccaggggtgtatgaggagccttgctaggcggggctagcgggaacacctcccctgtgctggccacgctggcggaggcaggtgtgcctgtaggatgcggtgggcggcccagctttgcctcaggaaggaaggaaacgaaagaaccccttgcctgctcagtgctgaggccacagagggcaggtcccccgagtgagtgcgggggacgcttggctgctgtttagctccactgtggccatggggagacccagcctgggggtgctggccccctcccggaggccccgtgtcccagccactctgcctgatcggggctgtgtgtgctgttttacggctcaggtccaaagacagcgcctgccttttcatcagaggccatgcgtctccctgtgtttcagacgttggtgatcaaagtgtccggtgtgatcctgtccgtggtcgggggcctggccgtgggaaaggtaacaaagtgcacatggccactccagctggagcctgaggccgccgggcccgcgagggccgccacgcccatgtgtgtctctgagcaccggtccttctggtctccaggaagggccgcgtcacgcgtgtctctgagcaccggtccttctggtctccaggaagggccgcgtcacgcgtgtctctgagcaccggtccttctggtctccaggaagggccgcgtcacgcgtgtctctgagcatcggtccttctggtctccaggaagggccgcgtcacgcgtgtctctgagcatcggtccttctggtctccaggaagggccgcgtcacgcgtgtctctgagcaccggtccttctggtctccaggaagggccgatgatccactcaggttcagtgattgccgccgggatctctcagggaaggtcaacgtcactgaaacgagatttcaaggtgagttgaaatcttgtgtgggtgggctccagatgccatgggcacgggcacgggcaccactcagggagatgggcttccctcagcacccccaggccgagagccccagccccatctgaggacagcctggcgggtggctcccagagccagcgggcacagtccctgcccggcaaggcctccctacggcccgctgcttccctccttgggtcccctgccacacgtgcatcagtgtttcccgtgggagggtctgtggctccaagcggcttctcagaggagtgcagaacctgagaccaagtgtgcccacctgttgtttatttgtcaagacacactttggaacactttttccccaaaaaagtccccagcatgttgatggggattgagctgcatttgtgtgtgattgtattttttttttttttttgagatggagtctctctgttgcccaggctggagtgcagtggtacaatctcagctcactgcagcctccacctcccaggttcaagcaattctcctgcctcagcatcccgagtagctgggattataggtgcccgccaccacgcctggctaagttttttgtatttttagtagagatggggttttgccatgttggccgggctggtctcaaactcccgacctcaggtgatccgcctgcctcggcctcccaaagtgctgggatgacaggcgtgaggcaccgcgccggccatgtgtgaatttagaggcaggcagcgtcccgcaggacaaagaacagcaaggctgggtttccatccgtgcgcttttcgttagagggtagaggtttttggaatcttgcgtgcgctggaaagtggagctcctggctgggtgtttgcgtgtttccctgggctgccggtggtggtgctgaccctgctgtctcttgccgtggtctgcagcacggtgctcttcaggaatcagagctgctgactcggttgtcctgaaagccccttcccctgcacagcccctgtcctggcagttgctctccctttctgagagccgtgccctcaaggaacctgccccgaccctggtctgtccctgttgcagatcttcgagtacttccgcagagacacagagaagcgggacttcgtctccgcaggggctgcggccggagtgtcagcggcgtttggagcccccgtgggtgaggagggccgcaccgggtccaatgctttgccctcgccctgtgtgttggaaggaacggtctcctctctgtaggcccagtgcccgctgagggtggcagaggcttggagtcacggccggggcatttggaagcggccggcagtgtacttgggtccagccctcagacctccctcagggtccctctctgtgtggctggggcccaccccattagcttctttctgacgtggtctgggttccctggagcctgggggagggagttggtggtgggcatggtgccctgtgtccagctggcacccgagccggccgcctgccttccaggtggggtcctgttcagcttggaggagggtgcgtccttctggaaccagttcctgacctggaggatcgtaagttcctgctgatggctgcctcctgatcagggtgcatgctgcgctctcatttcccaccatggggtccaccttggggccacccatcgagctgcggctggagctggaccccctgtgggtctgtcaggccttggtcctgcccaaagcagcggtcctgcctttgctgcccagttcgcccttggtcctgggcaccattgccagccctgggtggctcccgggtaggggatcaaacagccgggaacccagccctgccccaccttcccctcttgctctccactggcaagtccagagagggctgggccgctccttgcccgcacagtgcgcccacccctggctccagccccttcccttctgccttgggcggggtctgcagactcctggccccggggctgacaggaggggcgatggtccctgctggtccgtgagccctgggctgggagcgtggctctgagggcgctggtttcctgccctctgccgcagttctttgcttccatgatctccacgttcaccctgaattttgttctgagcatttaccacgggaacatgtgggacctgtccagcccaggcctcatcaacttcggaaggtttgactcggaggtaacctgccccatcgcccacctcgcccacctcgtatcctggtccaggaccctgtttgcttaaggcccaggttgagaatttggtcctttagaaaactctggttgatagctgtggagctgagagctcttgtgtaagctccagggccccgaggggctgcaggaagacaccccaagctgcccctcaggtcagggcaccatgtgaccagcagggcacctgggatgtcacacagttgctgcgtgcatggggcctcccacggcctgggggcacgtgcagcagccgctctcggggcaggtgggctcaggcctagtttccagggtagcctggggcctgggctggggagactctccgtgccatcgatagggcggctctgtgcgcaggaaactgggggaccacgggctatgttcccagtgcttggggccctccccgccccgggtgctgagggtggcagggtctctgagagcctcgctggccaccccgccaggcaggggccaggcctgctcagaacacccagtgtgtttctcccctgtggacttccgcagcctgcgtggaagggcgggaaggctctctgtggggacagctctcttaagatggtggtccttgagtttcagcagaaaggagctgtgggccttttccctcacatcctctgccttctccctctctctgcacagaaaatggcctacacgatccacgagatcccggtcttcatcgccatgggcgtggtgggtaagggcttctcccagcaccgcagggacggcctgcgggcctggctcagctgtgacgtggccatagagacgaggactggaggctgtggctccctggagcctgccctcatcccagggccacccgggggcctccagattcttccatgggcagtacacgtggggagtggggagcccaaagcttcgcttctgtggcttcccgttgtttatctctgttggcaaaaaccacagggctgcagggatggatgggatttcctgtaagagatagaattgctcccaccagtatttattgctctgctggacacctttgccctggaaggaaggcagagcctttgagaaacagctcccccagccctcagggtgtgatgatgtggaggaagcatcttaccaggaccccctagccccctgccgtccccttccctctgcaaaccctccagcttctcctgccatctgggagccggcgggcggaggcccgcacttttcctccggtgtcgctgactggcctttcccctgttcgcaggcggtgtgcttggagcagtgttcaatgccttgaactactggctgaccatgtttcgaatcaggtgaggagaaaccgcattgcatatcgcgttggcaggcgtggccacacaggccctttgaaagcggacgtggtggaatggggtttacactcctaggccacagccgaaagaaaggctgtgtatgcagcgtccttcctgatggtttccccggtggagctggtcagagatgtgtcccggggcctggagggtgacggactagcccaaggctaggagtgcgagggctcctggaggacggcccctgggtaggaagtgaggccctgcgtgggatcgggcctgggcgaggcatgcccaaccttcaccgcctggctctgcctggtagcaaccgcagctgtcctgggacaccggggccccccggcttcttccttcttggtctgtgctgatttcaatactgtcgggtacagccggggcacgggtagcgccacttcccacacatctggagaagttgctgccgaggagtctttaccccagggaagaggacgaccccaggacatttgggtgcctgattgatgattaaacacaggcctggccgggcgcggtgcctcacgactataatcccagcactttgggaggccgaggcgggtggatcacctgaggtcgggagttctagaccagcttgaccaacatggagaaaccccgtctctactaaaaaattcaaaaaaaaattagccagatgtagagccgggcgcctgtaatcccagctactcgggaggctgaggcaagacaattgcttgaacctgggaggtggaggttgcagtgagccaagatcgcagcactgcactccagcctgggcaacaagagcaaaactccgtctcaaaaacaaaaacaaacaaacaaaaagcaccacgggcccagtgtcctccatcagggactcgagttgccatggggcctgcggaggggccgcgctgccgtcctgcctgccatgcagcctgattcttggttccaggtacatccaccggccctgcctgcaggtgattgaggccgtgctggtggccgccgtcacggccacagttgccttcgtgctgatctactcgtcgcgggattgccagcccctgcaggggggctccatgtcctacccgctgcaggtgggaggctgggcccgggcggggtccagcaggcagggcagccacagggcggcctccaggaggctcgcttaggctacgggaggagggctgcccaccccgccgagttccagaagcgcatgggctggcgtgtctcaaagagggttagtcctgtccacccagatctcagaggaggccaggtgtctgctgaggtgccaggggaatgggcggtggtatgggggccagaggctccccccagtcctcttcccagaatggcagcctgacggggcgagcctcaggcgccctatgggggcaccatagatgtggacccaggagaaatgcaaacctccgtccacaactggacctgtgcctggcgctcacggctcaccgccgtccgtgcgtccatctgcactgtgacacggttgccctggaaagcactacgctcagaggaaccacacgtgaggtcacgcgacgtagccccattaacatgaaacatccagaacagggagagcctagaggcccagcagaccagtgggtgccacggcgggagtgggcaggatgggacgggtcaggtgtgaaccgttagagacgtgggaggcccggggccatggggttgaccagccttgctacactctgctccagccccgtggataacaccccctgtgctgctggagcccaggaggctctgggcctgtggcaccggggcgccaacagcctcttctaggagctcatgtgagcgcctgggcccaccttccccggcaccagggatggacagcgtctcagcccatggtcctgctaacccaccccccagggctagacacggccccctgctgggcctaggccgtgtgtgtcctcctttccctccgtgaccatggcttgggccttgtgtgtcctccttgccctctgtgaccgtggccctgacccaatggcaggatcgtgtggtttcgcgcctgatgctggccaggcacagggtacacggcctctcacggcgacaccaggtttgtgcctgcagcccaccagctcatctcccctcccaacgtgtgctctctcccgaccccacagctcttttgtgcagatggcgagtacaactccatggctgcggccttcttcaacaccccggagaagagcgtggtgagcctcttccacgacccgccaggtgtgtgtgggcagtgccgctgggcaggccctgggatcagggcctgggtgatgccttctggctgagtgtcccctgtgggctgaggttgcagccctgggctggggggtcatccctagcatggatcatagcagggactcactcctgtaatcccagcacttggagagaccaaggcaggaggatcacttgagcctaggaggttaagaccagcctgggcaacttagcgagactctgtctttgcaaaaaagcaacattatctggctacggtagtacacccacagtcccaggtacttgggaggctgggccgggaggattgcttgagcccagaaggttgaggccacaatgagctgtgattacatcactgcataccagcctgggtgacacagcgagaccctctctcaaaaaacaaaagaaaacccagcctggtgactcccacaccaagaccacggcctggcctcgctcgaccacaagtgtttcacggaagcgcagaccgcgaccttggagtgccggcctttcacctctgcagttgtgtccctggcggtctcacccgccctgcacgcagtacagtgctgcctgctccaggaaaggaaccccaggctgtggggggcaccctcttcccggagccaggctgcgagctgcaccacggtgcacacccatggagtgtagacctggcgctgctagacccagctgggccgccccgctggacgcggctcctgcttctgctggcatcagggccccgcagagcctcttcccctgtggcctccccatgggatccttttagcctttctgcttcccagggaggctgagaacagggagccttctggggaccgctgggctcgggagctcaggttgctgggctcctggaaggtgactgtgaggcccgagactgggcagcggggcagggcagtcctgcggaggcgggagtcgtggaggccccgtcagcccctcttctctcctaggctcctacaaccccctgaccctcggcctgttcacgctggtctacttcttcctggcctgctggacctacgggctcacggtgtctgccggggtcttcatcccgtccctgctcatcggggctgcctggggccggctctttgggatctccctgtcctacctcacgggggcggcggtgagtggggccggaggggaggctgtggggccctgcagctgagccaggtcttgcggcatcgcgggccggagcagaagtcccagggcaggacaaaagtgtcgcacctcacgtggttcacgggccgtgggcgttgtcctcgcgtggttcacgggccgtgggcgttgtcctgctgtggtggcagcgtgtactgtggcagcgcagcccatgtgtggagtctggaccaggcgaaggtagggggcggaggctcgtgtccttattcttgagaatgtgatgaaaagcagaggtgattgtgggctgctgcagagctgtttctagactccatggggtggatgtccggccagggctgctctctgtgaggccgggggccagagcggcatacactgccctccagacctcagcccccgcaggccttccttctctgcctgcctctgctgggactgggttctcttatgtgtcttctgtttctcatttcagtcgcttaaataagactgaaaacctgtaagaggccctggcaggaagcccccggccatgcttctcatccccggcaggaagcgcccactcctgctccccaggcccgtgtgctctgcccatctccctccgcacaagggtttggtttggtttttaaaattgaaacatgattcaaataccgtaaaactcatcgttttaaagagggcagttcagcggcgtttctcacgttcacgaggcagtgcggccgtcactaccacttctagaatgttccgtcatcccagaatggaaaccctgtgcccaccgaccctcgtgccccgctttctgcagcctccatgcctgggttctgtggcccagcctgatgttcccggggctctctgtgtcgtgtgtgccggggtttcactcctcatgctggacggtgctccctagttggcctgggctgctgcgtggtgactgtgccctctgcatcctccatgcctgccactcccctgttgctcgggtgctgagcgcctggttcaggccaaggatgcagcctccgcagcagggtgtactgtgctaggttgttctgtgtgtatgtacgcggccacgaggtttgttcctggctgtggggctgctgggcctgggcagggcctcctccgtctgtgtatcttggtgggtttgggcctgccaccacactgacacctcctccgtgtcacctcccacagatctgggcggaccccggcaaatacgccctgatgggagctgctgcccagctgggtatgtcccagctcttgcccgatgggtggggagctccacggggtctggagggggccatggctgtccttgcggggctagggtctgggagcaggtggatgggatgggtgctgcagagaaggcagtggccacgtgaccctgagccaggagggtggacgtgctggggttcatgatggctcccgcaggcgggattgtgcggatgacactgagcctgaccgtcatcatgatggaggccaccagcaacgtgacctacggcttccccatcatgctggtgctcatgaccgccaagatcgtgggcgacgtcttcattgaggtgcgccagggcctcgaagcctcaccctgagagcgtgggtgctgccataggggaggggcccctgtgagcctccaaacagccggtcccggggggtaggctcagggtttctgggggcggcctctgggctcccaggggtaggctcggggctccaggggtgggtgtggactcctcaagccctgtgttcccgccccgcccgcagggcctgtacgacatgcacattcagctgcagagtgtgcccttcctgcactgggaggccccggtcacctcacactcactcactgccaggtacagcgcccaggacacctgtgggtggggagggtgtccagcggcctcttgttgcacaggggcagggtgcacggcttgcgggctccaggcagccccgcgtttcctgtccagcggcttcacacctcaccagcccgcagaggtaactgtgggagttggtggcgtgtgacgggcatgtgtggccgggctcctccggcagggaggtgatgagcacaccagtgacctgcctgaggcggcgtgagaaggtcggcgtcattgtggacgtgctgagcgacacggcgtccaatcacaacggcttccccgtggtggagcatgccgatgacacccaggtaccgggcaccccatagacagggtcctgcctatgtgacctctgtcgagtccattggtgggaagcacacggcaaggtttgcaggatggggaagctgcacgtttgggtgcactggcagttccaggagtgccggaagctgagtgtgcagccatggagggctgtgtggacgctgaggctggtggggggggctgcggcctggcagggtcttggggttggcacccaggctgggctgagagccgtggcactgggggccgtgactttgtcaggaggccctgacaggacacacagctcggccactgctgtgtgtcttttagacgtggacactgggtgtttggaggttggtttttattgggacccagtggggctgcatctgccctgcagcaaagccaccatccctgggcccttggctctctgctgtgcgcggtcaggccccgctaccctgtcgccgatccttgggtcccgtggcattgtgcgtgtgggatgccatggcgaggctggtgtgagcaggtagccaccgacacggggcccatgcccagatgggaaatctggccggaacagggtcagagcggggcccgacacagcattccagcgcagcctcccaccctcgggcccgtggccctgaccgcgggcctgtcttgcagcctgcccggctccagggcctgatcctgcgctcccagctcatcgttctcctaaagcacaaggtgcgtgccaggctccgggccattgggcgggtgggggccccgggggtgctgcctgggtgcctgacacagggctctgccgcccgcaggtgtttgtggagcggtccaacctgggcctggtacagcggcgcctgaggctgaaggacttccgagacgcctacccgcgcttcccacccatccagtccatccacgtgtcccaggacgagcgggagtgcaccatggacctctccgagttcatgaacccctccccctacacggtgccccaggcatgtgcagggcatgggcatgggcgtggggcctgggactgaacagcagggggtggggtccagagcctcggggaggggcagccggggggggccacagcggagaggactcggtgactctgtctcctgtgaagggcctggcaggctttagagctgaagtcaaggggctgagggggctggccagacgggcgtgggggctcaggacgtgcctggacgccgtggtggggggtgcagggagccagcttgggtgagggtcccgcctgcctctgctgtgtgggcgggcactgacagctgtgcccctgctgcaggaggcgtcgctcccacgggtgttcaagctgttccgggccctgggcctgcggcacctggtggtggtggacaaccgcaatcaggtgagcgggg

SEQ ID NO: 3 - DNA, 28 bp, Artificial
  Primer
ttgcaggtcacatggtcggccgtcgctc

SEQ ID NO: 4 - DNA, 26 bp, Artificial
  primer
gacacgcggcgccgcagaaggctcac

SEQ ID NO: 5 - DNA, 22 bp, Artificial
  Primer
ccactccagctggagcctgagg

SEQ ID NO: 6 - DNA, 20 bp, Artificial
  Primer
gctgagggaagcccatctcc

SEQ ID NO: 7 - DNA, 20 bp, Artificial
  Primer
ttgcagtgagccaagatcgc

SEQ ID NO: 8 - DNA, 20 bp, Artificial
  Primer
ctcctcccgtagcctaagcg

SEQ ID NO: 9 - DNA, 450 bp, Homo sapiens
  misc_feature (1)..(450): Any 6 50 bp repeat units may be present or absent; represents 3-9 50 bp repeat units
gtgtctctgagcaccggtccttctggtctccaggaagggccgcgtcacgcgtgtctctgagcaccggtccttctggtctccaggaagggccgcgtcacgcgtgtctctgagcaccggtccttctggtctccaggaagggccgcgtcacgcgtgtctctgagcaccggtccttctggtctccaggaagggccgcgtcacgcgtgtctctgagcaccggtccttctggtctccaggaagggccgcgtcacgcgtgtctctgagcaccggtccttctggtctccaggaagggccgcgtcacgcgtgtctctgagcaccggtccttctggtctccaggaagggccgcgtcacgcgtgtctctgagcaccggtccttctggtctccaggaagggccgcgtcacgcgtgtctctgagcaccggtccttctggtctccaggaagggccgcgtcacgc

SEQ ID NO: 10 - DNA, 20 bp, Artificial
  Primer
tctagagcagggagcttgcg

SEQ ID NO: 11 - DNA, 20 bp, Artificial
  Primer
gccctggggccccactatct

SEQ ID NO: 12 - DNA, 20 bp, Artificial
  Primer
ccttggtgtcgggatgataa

SEQ ID NO: 13 - DNA, 20 bp, Artificial
  Primer
ggagtcagaggaggagggag

SEQ ID NO: 14 - DNA, 20 bp, Artificial
  Primer
gcacactgggcccttcataa

SEQ ID NO: 15 - DNA, 20 bp, Artificial
  Primer
ttcaccaagacccccaatcc

SEQ ID NO: 16 - DNA, 20 bp, Artificial
  Primer
gctgaggggctgcatctgtc

SEQ ID NO: 17 - DNA, 20 bp, Artificial
  Primer
aaggcaggcagccaagagag

SEQ ID NO: 18 - DNA, 20 bp, Artificial
  Primer
cagccactctgcctgatcgg

SEQ ID NO: 19 - DNA, 20 bp, Artificial
  Primer
aggctgtcctcagatggggc

SEQ ID NO: 20 - DNA, 20 bp, Artificial
  Primer
tcagagctgctgactcggtt

SEQ ID NO: 21 - DNA, 20 bp, Artificial
  Primer
aggaccaaggcctgacagac

SEQ ID NO: 22 - DNA, 20 bp, Artificial
  Primer
tcccctcttgctctccactg

SEQ ID NO: 23 - DNA, 20 bp, Artificial
  Primer
ctcaacctgggccttaagca

SEQ ID NO: 24 - DNA, 20 bp, Artificial
  Primer
aaggagctgtgggccttttc

SEQ ID NO: 25 - DNA, 20 bp, Artificial
  Primer
gtggcctaggagtgtaaacc

SEQ ID NO: 26 - DNA, 20 bp, Artificial
  Primer
ctcatctcccctcccaacgt

SEQ ID NO: 27 - DNA, 20 bp, Artificial
  Primer
cctcctgccttggtctctcc

SEQ ID NO: 28 - DNA, 20 bp, Artificial
  Primer
ctggaaggtgactgtgaggc

SEQ ID NO: 29 - DNA, 20 bp, Artificial
  Primer
tgaaccacgtgaggtgcgac

SEQ ID NO: 30 - DNA, 20 bp, Artificial
  Primer
tctgtgtatcttggtgggtt

SEQ ID NO: 31 - DNA, 20 bp, Artificial
  Primer
gggaacacagggcttgagga

SEQ ID NO: 32 - DNA, 20 bp, Artificial
  Primer
ggggtaggctcagggtttct

SEQ ID NO: 33 - DNA, 20 bp, Artificial
  Primer
cccaccaatggactcgacag

SEQ ID NO: 34 - DNA, 20 bp, Artificial
  Primer
catgcccagatgggaaatct

SEQ ID NO: 35 - DNA, 20 bp, Artificial
  Primer
cccggaacagcttgaacacc

SEQ ID NO: 36 - DNA, 20 bp, Artificial
  Primer
gggcctggcaggctttagag

SEQ ID NO: 37 - DNA, 20 bp, Artificial
  Primer
tccgggaggaaatgcagaag